(19) United States
(12) Patent Application Publication
(10) Pub. No.: US 2026/0183507 A1
(43) Pub. Date: Jul. 2, 2026

(54) METHOD FOR PROVIDING BIOFEEDBACK FOR PERSONALIZED MANAGEMENT, USER TERMINAL, AND METHOD OF CONTROLLING THE USER TERMINAL

(71) Applicant: Lee Sol Co.,Ltd., Seoul (KR)

(72) Inventors: Seung Woo Lee, Seoul (KR); Gusung Kwon, Seoul (KR); Hyuk Won, Seoul (KR)

(73) Assignee: Lee Sol Co.,Ltd., Seoul (KR)

(21) Appl. No.: 19/260,860

(22) Filed: Jul. 7, 2025

(30) Foreign Application Priority Data
Dec. 31, 2024 (KR) 10-2024-0202672
May 26, 2025 (KR) 10-2025-0068480

(51) Int. Cl. A61M 21/02; A61M 21/00; G16H 20/30; G16H 40/67

(52) U.S. Cl. CPC A61M 21/02; G16H 20/30; G16H 40/67; A61M 2021/0027; A61M 2021/0061; A61M 2021/0072; A61M 2021/0083; A61M 2205/3303; A61M 2205/3306; A61M 2205/3375; A61M 2205/502

(57) ABSTRACT

Provided is a personalized biofeedback providing method including: obtaining, through at least one of a terminal and a wearable biosensor, first information corresponding to at least one of Heart Rate Variability (HRV) information, electroencephalogram (EEG) information, heart rate information, temperature information, stress information, body composition information, weight information, oxygen saturation information, pulse information, blood pressure information, iris information, voice information, vein information, and electrocardiogram (ECG) information of a user; deriving a difference by comparing a first circadian rhythm, which is calculated based on a time point at which the first information is obtained through the terminal, with a preset reference circadian rhythm; and delivering a stimulus to a brain of the user to entrain synchronized oscillations in a plurality of regions of the brain of the user so that the first circadian rhythm matches the reference circadian rhythm, based on at least one of the terminal and a stimulation device.

### CROSS-REFERENCE TO RELATED APPLICATIONS

This application claims priority to and the benefit thereof under 35 U.S.C. § 119 to Korean Patent Application No. 10-2024-0202672 filed in the Korean Intellectual Property Office on Dec. 31, 2024, and Korean Patent Application No. 10-2025-0068480 filed in the Korean Intellectual Property Office on May 26, 2025, the entire contents of which are incorporated herein by reference.

### BACKGROUND

### (a) Field

This study was supported by the 2023 clinical trial (HI23C0607) on sleep improvement using tACS (transcranial alternating current stimulation) for sleep induction through brainwave modulation, funded by the Korea Health Industry Development Institute (KHIDI).

The disclosure relates to a method for providing biofeedback for personalized management, and more specifically, to a personalized biofeedback providing method that may support personalized management and biorhythm control by collecting and analyzing, in real time, a user's heart rate, sleep pattern, concentration data, and snoring sound, providing an optimal multi-modal therapy based on the individual's circadian rhythm and biometric data, automatically adjusting the intensity and type of therapy reflecting the user's real-time condition, and providing, via a user terminal, notifications on the optimal timing and recommended activities for sleep and concentration improvement.

### (b) Description of the Related Art

Generally, conventional sleep and concentration management solutions rely on user-specific device solutions, limiting accessibility for general users. In many cases, such solutions are not discoverable through specific keywords (for example, sleep-related terms) in app store searches, making user acquisition difficult.

In addition, the feedback on the usage results is neither specific nor useful, resulting in the user having to evaluate improvement effects based on subjective feeling. This lack of feedback lowers the reliability of the user experience and hinders continued usage.

Furthermore, conventional solutions rely on passive mode usage, requiring users to select and set modes such as sleep, healing, stress, and concentration based on their own knowledge. This makes it difficult for non-expert users to accurately select functions appropriate to their condition, leading to inefficient usage.

Moreover, in some popular applications, although the number of downloads and the paid conversion rate are high, the user experience deteriorates due to complex menu structures, sign-up procedures, and limited free services (for example, frequent paywall-inducing pop-ups, exposure to advertisements). In addition, users experience difficulty in navigating necessary menus, and lack convenience as they are required to go through unnecessary surveys and sign-up procedures to use the service.

Also, conventionally, functions such as sleep tracking, snoring detection, and sound therapy have been provided as basic services through applications, but such services have limited free features, and users are required to directly select menus and functions, resulting in inefficiency. Furthermore, there is a lack of additional personalized feedback or automated intervention solutions, and a limitation in that an automated personalized management centered on the user and an intuitive user experience are not provided.

In addition, recent neuroscience studies have revealed that abnormal interactions among brain networks related to various neuropsychiatric disorders and cognitive functions play an important role.

In this context, the Triple-Network Model has been proposed, which is based on the concept that the interactions among the Default Mode Network (DMN), Salience Network (SN), and Central Executive Network (CEN) play a central role in mental health and cognitive regulation.

There exist interactions and anticorrelations among the aforementioned networks.

That is, the SN plays a role in switching between the DMN and the CEN, and during the performance of specific tasks, the process in which the DMN is deactivated and the CEN is activated is important.

The DMN and the CEN have an anticorrelation relationship, in which the activity of the CEN decreases when the DMN is activated, and conversely, the activity of the DMN is suppressed when the CEN is activated.

If the SN does not function normally, the balance between the DMN and the CEN is disrupted, which may result in problems such as attention deficit, excessive internal thought (rumination), or executive dysfunction.

### SUMMARY

The disclosure is intended to solve the aforementioned problems by providing a personalized biofeedback providing method that may support personalized management and biorhythm control by collecting and analyzing, in real time, a user's heart rate, sleep pattern, concentration data, and snoring sound, providing an optimal multi-modal therapy based on the individual's circadian rhythm and biometric data, automatically adjusting the intensity and type of therapy reflecting the user's real-time condition, and providing, via a user terminal, notifications on the optimal timing and recommended activities for sleep and concentration improvement.

The problems to be solved described in the present application are not limited to those mentioned above, and may be extended to various matters derived from some embodiments described below.

According to an aspect of an embodiment, a personalized biofeedback providing method according to an embodiment may include: obtaining, through at least one of a terminal and a wearable biosensor, first information corresponding to at least one of Heart Rate Variability (HRV) information, electroencephalogram (EEG) information, heart rate information, temperature information, stress information, body composition information, weight information, oxygen saturation information, pulse information, blood pressure information, iris information, voice information, vein information, and electrocardiogram (ECG) information of a user; deriving a difference by comparing a first circadian rhythm, which is calculated based on a time point at which the first information is obtained through the terminal, with a preset reference circadian rhythm; and delivering a stimulus to a brain of the user to entrain synchronized oscillations in a plurality of regions of the brain of the user so that the first circadian rhythm matches the reference circadian rhythm, based on at least one of the terminal and a stimulation device, wherein the stimulus is one of: a first stimulus for entraining synchronized gamma oscillations in the plurality of regions of the brain of the user, a second stimulus for entraining synchronized delta, theta, alpha, or beta oscillations in the plurality of regions of the brain of the user, or a third stimulus that combines the first stimulus and the second stimulus.

In some embodiments, the reference circadian rhythm is divided into a wake-up zone within a first hour after waking up of the user, a sleep zone within a second hour before going to sleep of the user, and a daily zone excluding the wake-up zone and the sleep zone based on 24 hours, and wherein the difference varies depending on which of the divided zones the time point at which the first information is obtained falls into.

In some embodiments, when the EEG of the user is entrained by the stimulus, at least one of a parasympathetic nerve and a sympathetic nerve of the user is accelerated or decelerated by the EEG synchronization, whereby at least one of the HRV, temperature, and blood pressure of the user is changed so that the first circadian rhythm matches the reference circadian rhythm.

In some embodiments, when the first stimulus and the second stimulus are delivered together to the brain of the user, a region in which entrainment occurs is expanded, so that the time required to make the first circadian rhythm match the reference circadian rhythm is shortened, and wherein when transcranial Alternating Current Stimulation (tACS) is applied as one of electrical stimulation of the first stimulus, the tACS is a first combined signal in which ON/OFF is repeated according to a preset first frequency, and a signal that is ON according to the first frequency is modulated as a stimulus according to a preset second frequency.

In some embodiments, when the first stimulus and the second stimulus are delivered together to the brain of the user, a region in which entrainment occurs is expanded, so that the time required to make the first circadian rhythm match the reference circadian rhythm is shortened, and wherein when a binaural beats stimulus is applied as one of sound stimulations of the second stimulus, the binaural beats stimulus induces the entrainment through an auditory phenomenon in which the brain of the user recognizes a new frequency corresponding to a frequency difference by hearing sounds of different frequencies in both ears of the user.

In some embodiments, when the time point at which the first information is obtained is the wake-up zone, an alpha wave of 8 to 13 Hz is applied as the stimulus, when the time point at which the first information is obtained is the sleep zone, a delta wave of less than 4 Hz is applied as the stimulus, and when the time point at which the first information is obtained is the daily zone, at least one of the alpha wave of 8 to 13 Hz, the beta wave of 14 to 29 Hz, and the gamma wave of 30 to 100 Hz is applied as the stimulus.

In some embodiments, the method further includes: performing a wake-up mission of sound detection through the user terminal; and terminating an alarm of the user terminal when a sound corresponding to a preset sound is generated as a result of AI-based recognition of at least one sound among a clap sound, a laughing sound, and a speech sound of the user through the user terminal during the wake-up mission of sound detection.

In some embodiments, the method further includes: performing a wake-up mission of hand gesture recognition through the user terminal; and terminating an alarm of the user terminal when a preset specific action is taken based on recognition of a hand gesture of the user captured through the user terminal using Mediapipe-based gesture AI during the wake-up mission of hand gesture recognition.

In some embodiments, the method further includes: performing a wake-up mission of solving a math problem through the user terminal; and terminating an alarm of the user terminal when a preset correct answer is input based on recognition of input data entered through the user terminal using MLKit-based digital ink recognition AI during the wake-up mission of solving the math problem.

In some embodiments, the method further includes: performing a wake-up mission of answering a sleeping information quiz through the user terminal; and terminating an alarm of the user terminal when the input data entered through the user terminal corresponds to a preset correct answer for a question generated based on sleep record data of the user during the wake-up mission of answering the sleeping information quiz.

In some embodiments, the method further includes: analyzing circadian rhythm data of a user, and providing customized management feedback classified into a sleep mode, a wake-up mode, and a daily management mode based on a result of the analysis; or analyzing the circadian rhythm data of the user, calculating an optimal sleep start time and an optimal wake-up time for a next day based on an average sleep duration, a sleep start time, and a wake-up time pattern of the user, and providing a notification or recommendation message of the calculation result to a user terminal.

In some embodiments, the method further includes: presenting at least one sound therapy track for each sound therapy mode and executing a sound therapy track selected by the user; and presenting at least one piece of preset traditional fairy tale data and analyzing a traditional fairy tale selected by the user based on AI to provide the tale in a TTS (text-to-speech) service format.

In some embodiments, the method further includes: measuring a real-time concentration state of a user, classifying the concentration state into three stages, and automatically selecting and adjusting customized therapy content composed of at least one combination of sound therapy and electrical stimulation (tES) according to each of the stages to provide to the user.

In some embodiments, the method further includes: detecting snoring data of a user, and visualizing a snoring occurrence time to provide a notification to a user terminal.

In some embodiments, the detecting the snoring data of the user and visualizing the snoring occurrence time to provide the notification to the user terminal may include: providing snoring data to the user terminal through a notification icon displayed on the user terminal when snoring is detected.

In some embodiments, the method further includes: detecting a snoring sound based on an output of a neural network model for a sound received through a microphone; determining a snoring severity based on a frequency analysis result of the snoring sound; and providing a snoring reduction method when the snoring severity exceeds a preset value.

In some embodiments, the method further includes: determining an anteroposterior length and lateral length based on the frequency analysis result, and estimating an airway shape based on the anteroposterior length and the lateral length; determining a total variation norm, a formant concentration level, and a peak intensity position based on the frequency analysis result; estimating an obstruction level of the airway based on the total variation norm, the formant concentration level, and the peak intensity position; and determining the snoring severity based on the airway shape and the obstruction level.

In some embodiments, the determining the snoring severity based on the frequency analysis result of the snoring sound may include: determining the snoring severity based on the frequency analysis result of the snoring sound and an image analysis result for a head image of the user, wherein the head image includes a facial image and a cranial medical image.

In some embodiments, the determining the snoring severity based on the frequency analysis result of the snoring sound and the image analysis result for the head image may include: applying a first weight to the frequency analysis result, applying a second weight to the analysis result of the facial image, and applying a third weight to the cranial medical image to determine the snoring severity, wherein the third weight is greater than the second weight, and the second weight is greater than the first weight.

In some embodiments, the method further includes: recording a snoring occurrence time, duration, and snoring sound when the snoring severity exceeds a reference value; calculating a sleep score by applying different weights for each sleep stage to a user's sleep duration, the snoring occurrence time, and the duration; and providing the user's sleep duration, the snoring occurrence time, the duration, the snoring sound, and the sleep score through an alarm icon displayed on a screen.

In some embodiments, by activating a specific frequency of the brain and thereby reducing the influence of Default Mode Network (DMN), concentration may be effectively enhanced, providing an advantage of enabling high efficiency in various activities such as learning and work.

In addition, in some embodiments, based on the user's biometric data, fast activity may be induced during the day and slow waveforms may be induced at night to normalize a personalized circadian rhythm, thereby maintaining a balance between sleep and an awakened state and supporting an optimal lifestyle pattern.

Further, some embodiments, by activating working memory (WM) through rapid EEG activation, important learning content may be effectively stored in long-term memory, and the efficiency of WM during rest may be enhanced, thereby maximizing learning efficiency.

Additionally, in some embodiments, efficient sleep may be provided through personalized sleep management, thereby supporting successful long-term memory storage of learning content during the day and improving learning and memory capabilities.

Furthermore, in some embodiments, activities and rest during day and night may be personalized according to the user's circadian rhythm, thereby improving concentration, stress management, learning ability, and sleep efficiency simultaneously.

Also, in some embodiments, fast EEG and slow EEG may be activated depending on the situation, thereby supporting improvement in the harmonious functioning of the user's body and brain by maximizing productivity during the day and inducing deep and efficient sleep at night.

In some embodiments, a user status may be analyzed in real time, and EEG and therapy content may be automatically adjusted, enabling the user to experience an optimal solution without requiring separate settings.

In addition, in some embodiments, snoring sound may be analyzed in real time, and an optimal solution based on snoring severity may be provided.

In some embodiments, due to an intuitive interface and simple usage method, anyone may use it easily without complicated settings, and user satisfaction may be maximized through personalized management.

Also, in some embodiments, as an integrated solution supporting efficient sleep, memory enhancement, and improved concentration, it may provide both health and learning performance to the user.

The effects of the present application are not limited to the above-mentioned matters and should be understood to be extendable to various contents derived from the detailed description of some embodiments provided below.

### BRIEF DESCRIPTION OF THE DRAWINGS

The above and other aspects, features, and advantages of certain embodiments of the present disclosure will become apparent from the following description taken in conjunction with the accompanying drawings, in which:

FIG. 1 is a diagram illustrating an example of an operating environment of a system according to an embodiment.

FIG. 2 is a block diagram illustrating the internal configuration of a computing device 200 according to an embodiment.

FIG. 3 is a flowchart showing a method for providing personalized biofeedback according to an embodiment in a sequence of steps.

FIGS. 4A to 4C are diagrams illustrating a sleep recording function.

FIGS. 5A and 5B are diagrams illustrating a wake-up-by-hand-gesture function.

FIG. 6 is a diagram illustrating a wake-up-by-calculation function.

FIG. 7 is a diagram illustrating a wake-up-by-sleep-information function.

FIG. 8 is a diagram illustrating a case in which a user terminal detects a snoring sound according to an embodiment.

FIGS. 9A, 9B, 10A, 10B, 11A, and 11B are diagrams illustrating cases in which a user terminal performs frequency analysis on snoring sound according to an embodiment.

FIGS. 12A and 12B are diagrams illustrating cases in which a user terminal estimates a shape of an airway according to an embodiment.

FIG. 13 is a diagram illustrating a case in which a user terminal estimates an obstruction level of an airway according to an embodiment.

FIG. 14 is a diagram illustrating a case in which a user terminal outputs a snoring reduction method according to an embodiment.

FIGS. 15 to 16 are diagrams illustrating cases in which a user terminal outputs a message guiding a snoring reduction method according to an embodiment.

FIG. 17 is a diagram illustrating a case in which a user terminal determines a snoring severity by additionally using a head image according to an embodiment.

FIG. 18 is a diagram illustrating an example of a head image.

FIGS. 19A and 19B are diagrams illustrating cases in which a user terminal determines a snoring source location based on frequency analysis of snoring sound according to an embodiment.

FIG. 20 is a flowchart illustrating a case of guiding a snoring reduction method as part of a control method for a user terminal according to an embodiment.

### DETAILED DESCRIPTION OF THE INVENTION

Hereinafter, specific details for implementing the disclosure will be described in detail with reference to the accompanying drawings. However, in the following description, detailed descriptions of well-known functions or configurations that may unnecessarily obscure the gist of the disclosure will be omitted.

In the accompanying drawings, the same or corresponding components are given the same reference numerals. In addition, in the following description of the disclosure, redundant descriptions of the same or corresponding components may be omitted. However, the omission of the description regarding certain components does not intend to imply that such components are not included in the disclosure.

Advantages and features of the disclosure, and methods for achieving them, will become apparent with reference to the embodiments described below together with the accompanying drawings. However, the disclosure is not limited to the embodiments set forth below but may be implemented in various different forms, and these embodiments are provided merely to ensure completeness of the disclosure and to fully convey the scope of the invention to those skilled in the art.

The terms used in the disclosure will be briefly explained, and the disclosure will be specifically described. The terms used in the disclosure have been selected as general terms currently widely used, considering the functions in the disclosure, however, they may vary depending on the intention of a person skilled in the art, judicial precedents, or the emergence of new technologies. Also, in specific cases, there may be terms arbitrarily selected by the applicant, in which case the meaning will be clearly described in the relevant part of the disclosure. Therefore, the terms used in the disclosure should be defined based not merely on their names but based on their meanings and the overall content of the disclosure.

In the disclosure, singular expressions shall include plural expressions unless clearly specified as singular in context. Likewise, plural expressions shall include singular expressions unless clearly specified as plural in context. When a part of the disclosure states that a component “includes” another component, it is to be understood that, unless expressly stated otherwise, the component does not exclude other components and may further include additional components.

When describing the embodiments of the disclosure, detailed descriptions of well-known configurations or functions may be omitted if they are deemed to obscure the essential features of the disclosure. Also, in the drawings, parts irrelevant to the description of the disclosure are omitted, and similar parts are denoted by similar reference numerals.

In the disclosure, when a component is described as being “connected to,” “coupled to,” or “joined to” another component, it may include not only a direct connection but also an indirect connection through another component in between. Also, when a component is said to “include (comprise)” or “have” another component, it means that other components may be further included unless specifically stated otherwise.

In the disclosure, terms such as “first,” “second,” and so on are merely used to distinguish one component from another and do not limit the order or importance of the components unless otherwise specified. Therefore, a first component in one embodiment may be referred to as a second component in another embodiment, and likewise, a second component in one embodiment may be referred to as a first component in another embodiment.

In the disclosure, distinct components are described to clearly explain their respective features and do not necessarily imply that they are separate. That is, multiple components may be integrated into a single hardware or software unit, or one component may be distributed and implemented across multiple hardware or software units. Accordingly, unless stated otherwise, such integrated or distributed embodiments are also within the scope of the disclosure.

In the disclosure, the term “network” may include both wired and wireless networks. In this case, the network may refer to a communication network through which data exchange is performed between devices and systems or between devices, and is not limited to a specific type of network.

The embodiments described in the disclosure may be entirely hardware-based, partly hardware-based and partly software-based, or entirely software-based. The terms “unit,” “device,” or “system” used in the disclosure refer to computer-related entities, which may be hardware, a combination of hardware and software, or software. For example, in the disclosure, a unit, a module, a device, or a system may be a running process, processor, object, executable file, thread of execution, program, and/or computer, but is not limited thereto. For example, both an application running on a computer and the computer itself may correspond to a unit, a module, a device, or a system in the disclosure.

Also, in the disclosure, the term “device” may refer not only to mobile devices such as smartphone, tablet PC, wearable device, and head mounted display (HMD), but also to fixed devices such as PC or home appliance with display function. As an example, the device may be a cluster inside a vehicle or an IoT (Internet of Things) device. That is, in the disclosure, a device may refer to any device on which an application can operate and is not limited to a specific type. For convenience of explanation, a device on which an application operates is referred to as a device.

In the disclosure, the communication method of the network is not limited, and connections between respective components may not be based on the same network type. The network may include not only communication methods utilizing communication networks (e.g., mobile communication networks, wired internet, wireless internet, broadcasting networks, satellite networks), but also short-range wireless communication between devices. For example, the network may include all communication methods by which objects can network, and is not limited to wired communication, wireless communication, 3G, 4G, 5G, or other methods. For example, the network may refer to a communication network by one or more communication methods selected from among LAN (Local Area Network), MAN (Metropolitan Area Network), GSM (Global System for Mobile Network), EDGE (Enhanced Data GSM Environment), HSDPA (High Speed Downlink Packet Access), W-CDMA (Wideband Code Division Multiple Access), CDMA (Code Division Multiple Access), TDMA (Time Division Multiple Access), Bluetooth, Zigbee, Wi-Fi, VoIP (Voice over Internet Protocol), LTE Advanced, IEEE802.16m, WirelessMAN-Advanced, HSPA+, 3GPP LTE (Long Term Evolution), Mobile WiMAX (IEEE 802.16e), UMB (formerly EV-DO Rev. C), Flash-OFDM, iBurst and MBWA (IEEE 802.20) systems, HIPERMAN, BDMA (Beam-Division Multiple Access), Wi-MAX (World Interoperability for Microwave Access), and communication using ultrasonic waves, but is not limited thereto.

The components described in various embodiments do not necessarily mean that they are essential components, and some may be optional components. Therefore, embodiments composed of a subset of the components described in the disclosure are also within the scope of the disclosure. Additionally, embodiments that further include other components in addition to those described in various embodiments are also within the scope of the disclosure.

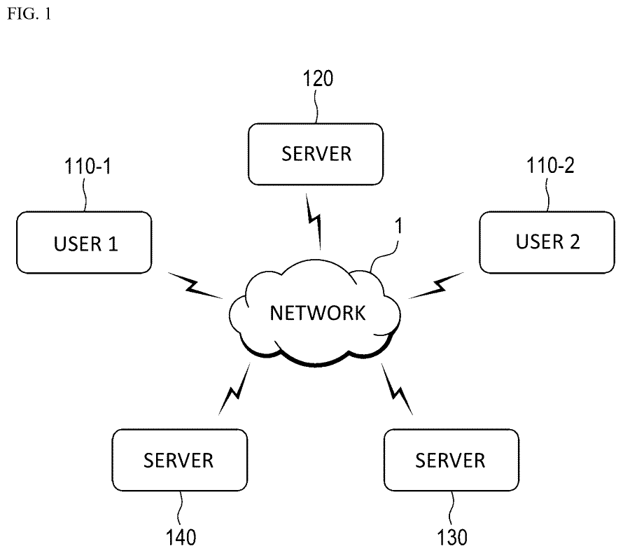

FIG. 1 is a diagram illustrating an example of an operating environment of a system according to an embodiment.

Referring to FIG. 1, an examinee device 110 and one or more servers 120, 130, and 140 are connected through a network 1. This FIG. 1 is merely an example for explaining an embodiment, and the number of examinee device or servers is not limited to that illustrated in FIG. 1.

The examinee device 110 may be a mobile terminal or a fixed terminal implemented as a computer system. For example, the examinee device 110 may be implemented as a smartphone, mobile phone, navigation device, computer, laptop, terminal for digital broadcasting, PDA (Personal Digital Assistant), PMP (Portable Multimedia Player), tablet PC, game console, wearable device, IoT (Internet of Things) device, VR (Virtual Reality) device, or AR (Augmented Reality) device. In an embodiment, the examinee device 110 may refer to one of various physical computer systems capable of communicating with other servers 120 to 140 through the network 1 using a wireless or wired communication method.

Each server may be implemented as a computer device or a plurality of computer devices that communicate with the examinee device 110 via the network 1 to provide commands, codes, files, content, services, and so on. For example, a server may be a system that provides respective services to the examinee device 110 connected through the network 1. In a more specific example, a server may provide, to the examinee device 110, a service (for example, information provision, etc.) intended by an application, through the application that is installed and executed on the examinee device 110 as a computer program. In another example, the server may distribute files for installation and execution of the above-described application to the examinee device 110 and may receive examinee input information to provide a corresponding service.

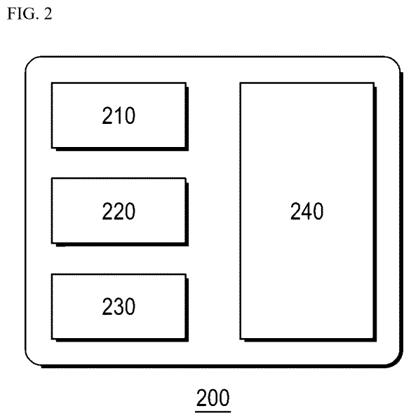

FIG. 2 is a block diagram illustrating an internal configuration of a computing device 200 according to an embodiment.

Referring to FIG. 2, the computing device 200 may be applied to the examinee device 110 or the servers 120 to 140 as described above with reference to FIG. 1, and each device and each server may have the same or similar internal configuration by including or excluding certain components.

In addition, the computing device 200 may include a memory 210, a processor 220, a communication module 230, and a transceiver 240. The memory 210 may be a non-transitory computer-readable recording medium and may include permanent mass storage devices such as RAM (random access memory), ROM (read only memory), a disk drive, an SSD (solid state drive), and flash memory. Here, non-transitory permanent mass storage devices such as ROM, SSD, flash memory, and disk drive may be separate permanent storage devices distinguished from the memory 210 and may be included in the above-described device or server.

In addition, the memory 210 may store an operating system and at least one program code (for example, code for a browser installed and executed on the examinee device 110 or an application installed on the examinee device 110 for providing a specific service). These software components may be loaded from a computer-readable recording medium separate from the memory 210. Such separate computer-readable recording media may include floppy drives, disks, tapes, DVD/CD-ROM drives, memory cards, and other computer-readable recording media.

In another embodiment, software components may be loaded into the memory 210 via the communication module 230 instead of from a computer-readable recording medium. For example, at least one program may be loaded into the memory 210 based on a computer program (for example, the above-described application) that is installed via files provided by a file distribution system (for example, the above-described server) that distributes installation files of applications or files created by developers through the network 1.

The processor 220 may be configured to process commands of a computer program by performing basic arithmetic, logic, and input/output operations. The commands may be provided to the processor 220 by the memory 210 or the communication module 230. For example, the processor 220 may be configured to execute commands received according to program code stored in a storage device such as memory 210.

The communication module 230 may provide a function for enabling communication between the examinee device 110 and the servers 120 to 140 through the network 1, and may provide a function for each of the examinee device 110 and/or the servers 120 to 140 to communicate with another electronic device.

The transceiver 240 may be a means for interfacing with external input/output devices (not shown). For example, the external input device may include devices such as a keyboard, a mouse, a microphone, or a camera, and the external output device may include devices such as a display, speaker, or haptic feedback device.

In another example, the transceiver 240 may also be a means for interfacing with a device that integrates input and output functions into one, such as a touchscreen.

Additionally, in some embodiments, the computing device 200 may include more components than those of FIG. 2 depending on the nature of the applied device. For example, when the computing device 200 is applied to the examinee device 110, it may be implemented to include at least some of the above-mentioned input/output devices or may further include other components such as a transceiver, a GPS (Global Positioning System) module, a camera, various sensors, a database, and the like.

In a more specific example, when the examinee device is a smartphone, it may be implemented to further include various components generally included in a smartphone, such as an accelerometer, a gyroscope, a camera module, various physical buttons, buttons using a touch panel, input/output ports, and a vibrator for vibration.

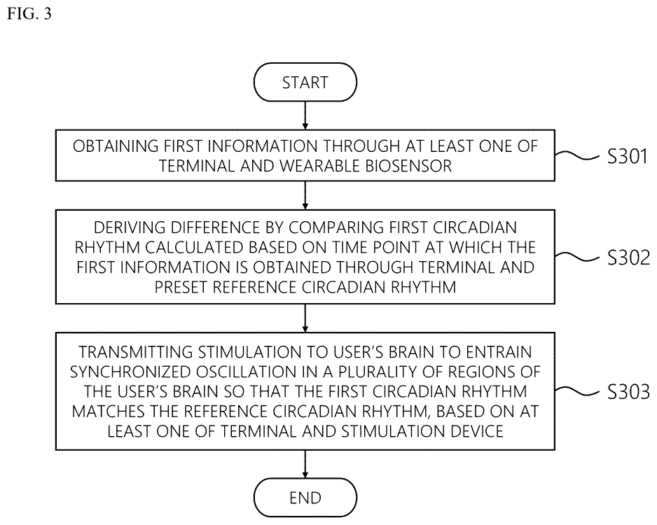

FIG. 3 is a flowchart illustrating a personalized biofeedback providing method according to an embodiment.

Referring to FIG. 3, the personalized biofeedback providing method according to an embodiment may include: a first step (S301) of obtaining first information, which is at least one among Heart Rate Variability (HRV) information, electroencephalogram (EEG) information, heart rate information, temperature information, stress information, body composition information, weight information, oxygen saturation information, pulse information, blood pressure information, iris information, voice information, vein information, and electrocardiogram (ECG) information of a user, through at least one of a terminal and a wearable biosensor; a second step (S302) of deriving a difference by comparing a first circadian rhythm calculated based on a time point at which the first information is obtained through the terminal and a preset reference circadian rhythm; and a third step (S303) of transmitting stimulation to the user's brain to entrain synchronized oscillation in a plurality of regions of the user's brain so that the first circadian rhythm becomes the same as (or matches) the reference circadian rhythm, based on at least one of the terminal and a stimulation device.

In step S301, biometric information of the user is obtained using a wearable biosensor. The wearable biosensor may be a brain stimulation device.

The wearable biosensor may include electrodes capable of measuring EEG in real time, and may be configured to include: an EEG sensor that is in direct contact with the user's scalp; a tACS stimulation module in which electrodes for tACS are integrated with the EEG sensor; a processor that controls EEG data collection and tACS signal generation; an integrated control module through which real-time data analysis and stimulation adjustment are performed; a wireless communication module for exchanging data with a terminal such as a smartphone via Bluetooth or Wi-Fi; and a power supply designed as a battery-based power supply unit for portability. In addition, the wearable biosensor may include a flexible band structure designed to adhere closely to the user's scalp, and may apply a lightweight and ergonomic design to prevent discomfort during long-term wearing.

In step S302, the current state of the user is analyzed to calculate the first circadian rhythm, and the difference may be derived by comparing it with the reference circadian rhythm.

The first process of step S302 is to express the current state of the user as a circadian rhythm based on biometric data collected via the wearable biosensor.

To this end, the disclosure classifies various data collected in step S301, such as HRV, EEG, heart rate, stress index, body temperature, and oxygen saturation, by time zones. Here, the time zones may be divided into three sections based on 24 hours. For example, the wake-up zone may be within one hour after waking up, the sleep zone may be within two hours before sleeping, and the daily zone may be the remaining time excluding the wake-up and sleep zones.

In step S302, the disclosure may evaluate whether the user is in a sufficiently awakened state by using the alpha wave activation level and heart rate data collected in the wake-up zone.

In addition, data collected in real time from the wearable biosensor may be expressed as the first circadian rhythm reflecting the current state of the user through a signal processing and analysis process. For example, if the user's EEG data shows excessive activation of delta waves (related to sleep), this may indicate that the user has not transitioned from sleep to an awakened state.

Also, in step S302, the reference circadian rhythm may be defined. Here, the reference circadian rhythm is a value that defines an ideal state based on scientific studies.

For example, the wake-up zone may be a zone in which the alpha wave (8 to 13 Hz) is activated and the user smoothly transitions to an awakened state; the sleep zone may be a zone in which the delta wave (4 Hz) is dominantly activated to maintain a deep sleep state; and the daily zone may be a zone in which the gamma wave (30 to 80 Hz) and beta wave (14 to 29 Hz) are appropriately combined to enhance concentration and productivity. In addition, auxiliary data such as heart rate, HRV, and stress index in each zone may also be set as reference values.

Also, in step S302, the disclosure may derive how much the user's current state (first circadian rhythm) analyzed from the wearable biosensor differs from the reference value. For example, if the alpha wave in the wake-up zone is below the reference value (8 to 13 Hz) and the delta wave is excessively activated, it may be evaluated that the user is in a low awakened state.

In addition, in step S302, the difference in each data set may be quantified to numerically represent the extent to which the current state of the user deviates from the reference state. This quantitative difference may be used as a core input value for the stimulation design provided in the following step (S303).

Moreover, step S302 performs a detailed time-zone-based analysis of the difference between the current state of the user and the reference rhythm.

For example, if the alpha wave activation is low and the stress index is high in the wake-up zone, the user may be evaluated as not sufficiently awakened. Also, if the delta wave activation is low and HRV is high in the sleep zone, the user may be evaluated as not having entered deep sleep. Furthermore, if the gamma wave and beta wave are inactivated or the stress index is high in the daily zone, it may be evaluated that the user has reduced concentration and productivity.

In addition, in step S302, the disclosure transmits the data quantifying the difference between the first circadian rhythm and the reference circadian rhythm to step S303. This data is used as core information for designing personalized stimulation, and in step S303, personalized stimulation such as tACS, binaural beats, or light stimulation is provided in order to reduce such difference.

In step S303, based on the difference derived in step S302, stimulation may be delivered to the user's brain to entrain synchronized oscillation in a plurality of regions of the user's brain so that the user's first circadian rhythm becomes the same as the reference circadian rhythm. In this step, stimulation such as electrical stimulation (tACS), binaural beats, or light stimulation may be delivered to the user's brain using the wearable biosensor, particularly in a manner that induces synchronized oscillation (e.g., EEG) in a plurality of regions of the brain.

More specifically, step S303 adjusts the user's first circadian rhythm to be the same as (or substantially the same as, or similar to) the reference circadian rhythm by providing personalized stimulation using the wearable biosensor. In this process, a combined signal including tACS and binaural beats may be used to induce neural entrainment and EEG synchronization, and stimulation may be finely designed and applied depending on the user's condition.

tACS operates such that electrical stimulation are turned on (ON) and off (OFF) repeatedly based on a preset first frequency, and this signal is modulated according to a second frequency to apply stimulation to specific regions of the brain.

For example, when the user is in the wake-up zone, tACS stimulation for activating alpha wave (8 to 13 Hz) may be applied to specific brain regions including the frontal lobe. In the sleep zone, stimulation of delta wave (below 4 Hz) may be applied to the occipital lobe to induce a deep sleep state. In the daily zone, activation of gamma wave (30 to 100 Hz) or beta wave (14 to 29 Hz) may be induced to improve concentration and productivity. Such stimulation analyzes the user's EEG data in real time to provide optimized intensity and frequency, and the preset signal pattern focuses on enhancing synchronization of neuronal firing.

The binaural beats stimulus induces brain synchronization by delivering sound signals of different frequencies to both ears of the user, so that the brain perceives the frequency difference between the two signals and entrains to the new frequency. For example, when 200 Hz is delivered to one ear and 210 Hz to the other, the brain recognizes the frequency difference (10 Hz) as a new frequency, which acts as stimulation for activating the alpha wave. In the wake-up zone, binaural beats designed to target alpha wave (8 to 13 Hz); in the sleep zone, delta wave (0.5 to 4 Hz); and in the daily zone, gamma wave (30 to 100 Hz) or beta wave (14 to 29 Hz) may be used to adjust the user's EEG state.

In addition, in step S303, the combined signal may be applied by merging tACS and binaural beats to enhance the effects of both stimulation methods. For example, when stimulation for gamma wave (40 Hz) is required, the disclosure may simultaneously provide electrical stimulation of tACS and 40 Hz binaural beats to enhance the EEG synchronization effect through interaction of physical and sensory stimulations. Such stimulation may be applied simultaneously to a plurality of regions of the brain to adjust the connectivity of neural circuits and the balance of EEG.

Moreover, the disclosure may monitor the user's EEG data via the wearable biosensor through a real-time neurofeedback system, and may dynamically adjust stimulation settings (e.g., frequency, intensity, pattern, etc.) by continuously evaluating the neural state during stimulation. For example, when the target level of alpha wave activation is not reached, the disclosure may adjust the current intensity of tACS or the frequency of the binaural beats to maximize the stimulation effect. This feedback loop ensures accuracy of stimulation and provides the user with optimized EEG synchronization.

Furthermore, the disclosure may provide a wake-up mission suitable for the current state of the user, such as sound detection, hand gesture recognition, solving a math problem, and the like.

More specifically, the disclosure may cause one of the wake-up missions, including sound detection, hand gesture recognition, solving a math problem, and answering a sleeping information quiz, to be performed via the user terminal.

When the wake-up mission of sound detection is performed, the disclosure may recognize one or more sounds among a user's clapping sound, laughing sound, or voice through the user terminal based on AI, and when the recognized result matches a preset sound, it is determined that the wake-up mission of sound detection has been completed, and the alarm of the user terminal is terminated.

When the wake-up mission of hand gesture recognition is performed, the disclosure may recognize the user's hand gesture captured through the user terminal using Mediapipe-based gesture AI, and when the recognized result corresponds to a preset specific action, it is determined that the wake-up mission of hand gesture recognition has been completed, and the alarm of the user terminal is terminated.

When the wake-up mission of solving a math problem is performed, the disclosure may recognize input data entered via the user terminal using MLKit-based digital ink recognition AI, and when the recognized result matches a preset correct answer, it is determined that the wake-up mission of solving the math problem has been completed, and the alarm of the user terminal is terminated.

When the wake-up mission of answering a sleeping information quiz is performed, the disclosure may present a question generated based on the user's sleep record data, and when the input data entered via the user terminal corresponds to a preset correct answer, it is determined that the wake-up mission of answering the sleeping information quiz has been completed, and the alarm of the user terminal is terminated.

In addition, in some embodiments, the disclosure may analyze the user's circadian rhythm data and calculate the optimal sleep start time and wake-up time for the following day based on the user's average sleep duration, sleep start time, and wake-up time pattern, and may provide the calculation result as a notification or recommendation message to the user terminal.

In addition, in some embodiments, in the case of the user terminal, to enable automatic connection with the wearable biosensor, the terminal may operate based on a BLE (Bluetooth Low Energy) protocol, and may include a Bluetooth module that supports continuous data connection with low power consumption, a Wi-Fi module that may be used in parallel for certain high-speed data processing, and a device discovery algorithm for scanning nearby wearable devices and identifying MAC addresses.

Additionally, the wearable biosensor may include an embedded Bluetooth module equipped with a low-power Bluetooth chipset for transmitting and receiving sensor data to and from the user terminal; a sensor processor that includes a chipset (for example, an ARM Cortex-M series) for collecting and processing sensor data (e.g., heart rate, temperature, etc.); and a power management circuit that optimizes power consumption when switching the device from sleep mode to active mode.

At this time, the user terminal may activate the BLE module to search for wearable sensors in the advertising state and estimate the physical distance based on the sensor's unique MAC address and signal strength.

The wearable biosensor periodically broadcasts advertise packets to inform nearby devices of its presence, and the packets may include the device name, service UUID, data format information, and the like.

The user terminal identifies a suitable device nearby based on the sensor's UUID, then initiates the initial connection procedure, and during this process, performs device-to-device authentication according to BLE security procedures through encryption key exchange to ensure secure data communication.

Once the connection between the user terminal and the wearable biosensor is completed, a data communication channel is established using the GATT (Generic Attribute Profile) protocol of BLE, and data characteristics of the wearable sensor (e.g., heart rate, oxygen saturation, etc.) are defined as GATT characteristics, which are subscribed to by the user terminal.

In addition, data measured by the wearable biosensor is transmitted to the terminal in real time via Bluetooth, and in this process, the data transmission interval may be optimized by utilizing the low-power characteristics of BLE. Furthermore, the integrity of the transmitted data may be verified by a CRC (Cyclic Redundancy Check) function built into the sensor hardware.

Regarding the automatic connection process, after the initial connection, the user terminal and the wearable biosensor may store pairing information through persistent bonding, and in subsequent connections, connection may proceed immediately by skipping the advertising and authentication process.

In addition, when the user terminal is within a specific range, the wearable biosensor may be automatically activated from sleep mode through a wake-up signal. At this time, the power management unit of the wearable biosensor may supply minimum power only to the BLE module to extend battery life.

Furthermore, the user terminal may automatically attempt reconnection based on the stored MAC address and the UUID of the sensor, and upon successful connection, the sensor data stream may automatically resume.

Once the connection is completed, the data collected from the wearable biosensor is transmitted to a dedicated application of the user terminal, and the user terminal performs data analysis and updates the U in real time so that the user may check the data in real time. The analyzed data may also be uploaded to a cloud server through the user terminal and may be used for long-term pattern analysis and personalized feedback provision.

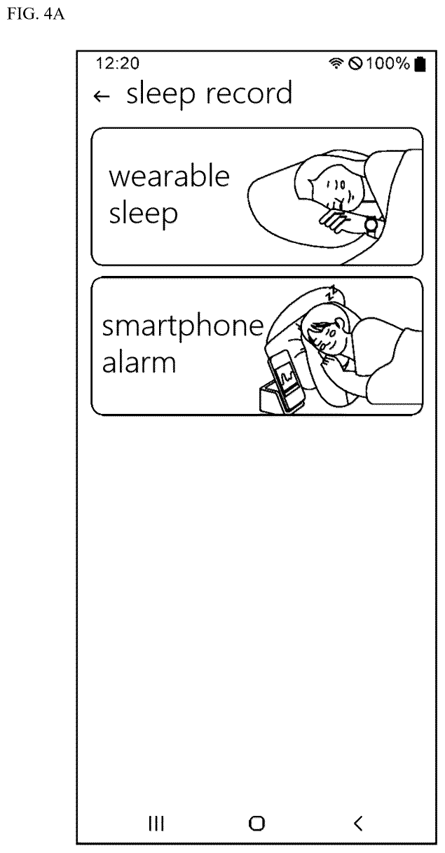

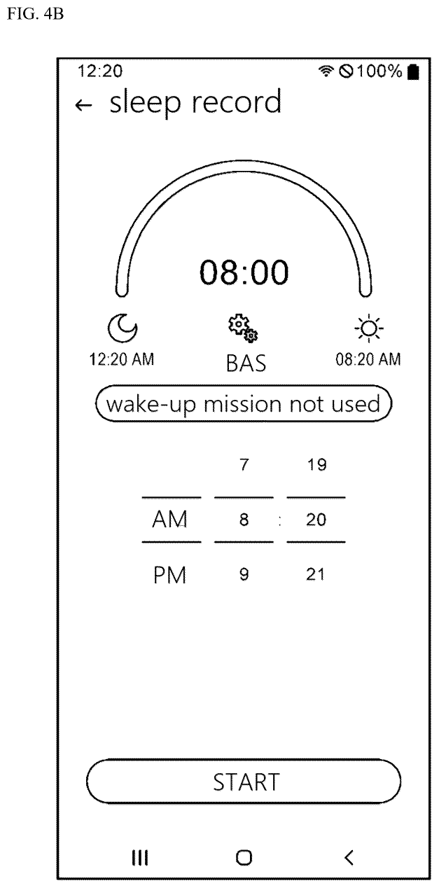

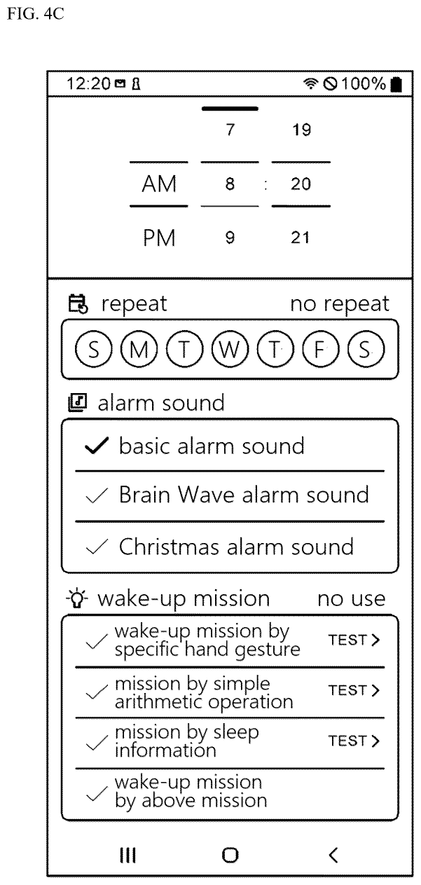

FIGS. 4A to 4C are diagrams illustrating a sleep recording function.

Referring to FIG. 4A, a wearable sleep item is an item for collecting sleep data through a wearable device such as a smartwatch, and a smartphone alarm item is an item for collecting sleep data and executing an alarm function through a sensor of a user terminal (for example, a smartphone, etc.).

In the wearable sleep item, when the user wears a smartwatch (e.g., Galaxy Watch) during sleep, heart rate, movement, and sleep stages (light sleep, deep sleep, REM sleep) may be detected through the wearable device. At this time, the user may synchronize the smartwatch with a dedicated application so that the sleep data is automatically recorded in the app.

Meanwhile, when the user selects the smartphone alarm item, the screen transitions to an alarm setting screen such as in FIG. 4B.

At this time, the user may set a desired wake-up time in “hour” and “minute” units, and the wake-up time is displayed by distinguishing between AM and PM.

In addition, on this screen, the user may freely select from among a basic alarm sound (BAS in the figure), a special alarm sound, a brain wave alarm sound, and an event alarm (for example, a Christmas alarm), and may also set a wake-up mission.

When the user selects the “wake-up mission not used” button, the screen transitions to a wake-up mission setting screen such as in FIG. 4C.

The wake-up mission setting screen is a function that allows configuration such that a specific action or task should be performed by the user when the alarm rings in order for the alarm to be dismissed.

At this time, types of wake-up missions may include wake-up by sound, wake-up by hand gesture, wake-up by solving a math problem, wake-up by sleep information, and wake-up by random mission.

Wake-up by sound corresponds to the wake-up mission of sound detection. In this case, the user terminal may recognize one or more sounds among the user's clapping sound, laughing sound, and voice using AI, and when the recognized result matches a preset sound, the alarm of the user terminal is terminated as it is determined that the wake-up mission of sound detection has been completed.

More specifically, in the case of the wake-up mission of sound detection, the user is required to make a specific sound to perform the mission, and if the sound is successfully recognized through AI-based sound recognition technology, the alarm is dismissed.

For example, the user may select one of the missions from the smartphone alarm setting menu, and when the alarm rings, the selected mission is displayed on the screen in the form of a popup. At this time, the microphone of the user terminal detects the sound generated by the user in real time, and the AI algorithm compares the collected sound data with sound patterns corresponding to an IDX. In this process, when the user makes a sound, the AI analyzes it in real time to determine whether the mission has been completed, and if the mission is successful, the alarm is automatically dismissed.

If the sound is not properly recognized, a message such as “Please try again” is displayed on the user terminal, and in this case, the user may perform the mission again or select an alternative mission.

In this process, the disclosure may guide the user to say a specific phrase (for example, “Good morning”), detect the naturalness of the user's voice and the accuracy of pronunciation, or analyze the rhythm and tone of laughter through a mission such as “Please laugh.” In addition, the disclosure may detect the clapping sound generated in the high-frequency range through a mission such as “Please clap your hands.”

Next, wake-up by hand gesture will be described in more detail below.

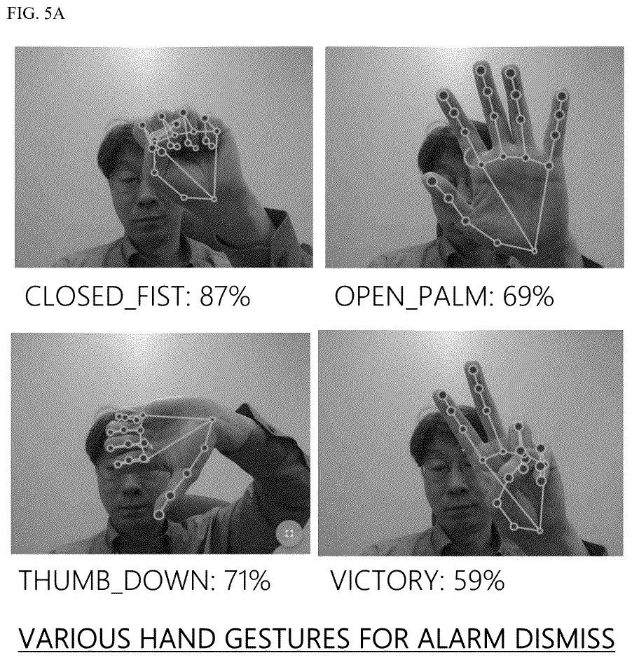

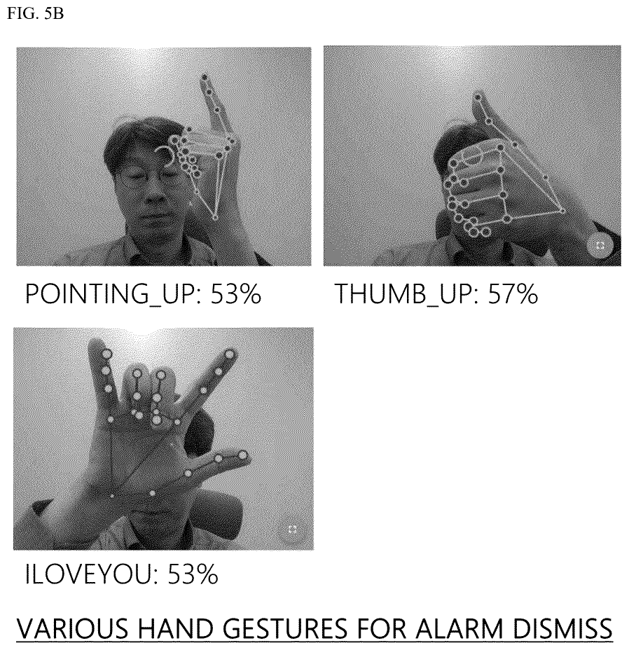

FIGS. 5A and 5B are diagrams illustrating a wake-up-by-hand-gesture function.

Referring to FIGS. 5A and 5B, the wake-up mission of hand gesture utilizes Gesture AI technology of Google Mediapipe to induce the user to perform a specific hand gesture, recognize the gesture, and release the alarm. This mission enables the wake-up process to be performed more actively and enjoyably through user interaction.

To this end, when the mission starts, the camera of the user terminal is activated, and the user's hand gesture is captured in real time. The Mediapipe-based gesture AI analyzes the landmarks of the hand (e.g., finger joint positions) captured in real time.

In addition, the gesture AI, Mediapipe, classifies the hand gesture into specific labels (for example, “thumbs up,” “victory,” etc.), and each gesture is stored with a predefined ID value, based on which the accuracy is evaluated.

The gestures may include a variety of types, such as making a fist, fully spreading fingers, pointing upward with the index finger, thumbs up, thumbs down, making a V-shaped victory pose, and making the “I love you” hand gesture.

When the alarm rings, the user activates the camera screen of the user terminal to perform the hand gesture, and a target gesture and its instruction (for example, “Please give a thumbs up”) are displayed at the top of the screen.

In addition, the AI analyzes the user's hand gesture in real time, and if the gesture matches the target, a success signal is output, and if the required accuracy is satisfied for a certain period of time, the alarm may be dismissed.

Conversely, if the user fails to perform the designated action, the process may proceed to the next stage or output an additional notification.

This allows not only for simple movement execution but also for awakening the user's brain through physical activity, and adds a fun element through a gamified experience, thereby increasing the user's willingness to wake up. By combining technical accuracy with user experience, a more enjoyable and effective wake-up solution may be provided.

Next, wake-up by solving a math problem will be described in more detail below.

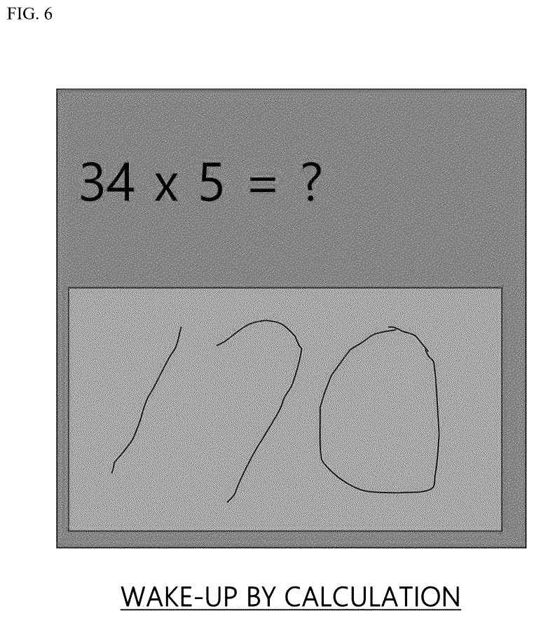

FIG. 6 is a diagram illustrating a wake-up-by-solving-a-math-problem function.

Referring to FIG. 6, in the case of the wake-up mission of solving a math problem, the user is induced to directly input the answer by hand, and a function is provided that analyzes the user's handwritten input using MLKit-based digital ink recognition AI.

For example, when the alarm rings, a math problem (for example, “34×5=?”) is displayed at the top of the screen of the user terminal. In this case, the user is required to solve the problem and input the correct answer directly in order for the alarm to be dismissed.

In addition, an input area is displayed on the screen of the user terminal, and the user inputs the answer directly using a finger or stylus pen. At this time, the input area analyzes the user's input and converts it into text using MLKit digital ink recognition technology.

Furthermore, the given problem may be composed of simple arithmetic operations (e.g., addition, subtraction, multiplication, division, or their combination), or it may be freely set at a difficulty level that the user may solve quickly. In addition, based on AI, the user's handwritten answer is converted into digital format and compared with the correct answer to determine whether the mission is successful. When the correct answer is input, the alarm is dismissed; otherwise, a retry may be requested.

Through this, the disclosure may activate the user's brain by simultaneously requiring mathematical thinking and hand gesture, and may quickly induce the user into an awakened state. Through a gamified wake-up process, instead of simply turning off the alarm, the wake-up process may be made enjoyable through an engaging task in which the user actively participates.

Next, wake-up by sleep information will be described in more detail below.

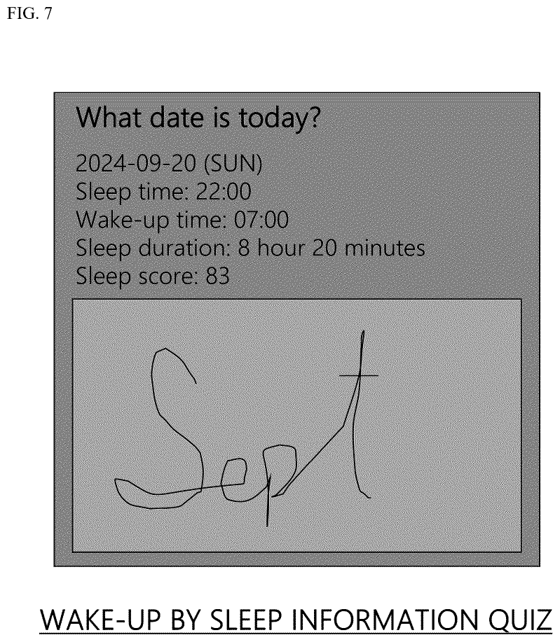

FIG. 7 is a diagram illustrating a wake-up-by-sleep-information function.

Referring to FIG. 7, in the case of the wake-up mission of answering a sleeping information quiz, the disclosure may make the wake-up process more interactive by utilizing the user's sleep data and induce the user's memory and information input.

To this end, the screen of the user terminal displays the sleep data recorded during the user's sleep, and a question is automatically generated based on specific information within the provided sleep data.

For example, a question such as “What date is today?” or “What time did you fall asleep yesterday?” may be generated and displayed on the screen of the user terminal.

In addition, a digital handwriting input area may be provided on the screen of the user terminal for the user to input a response to the corresponding question using a finger or stylus pen.

Once the user's response is entered, the MLKit-based digital ink recognition AI converts the user's handwritten input into text and determines whether the input matches the correct answer. If the input is correct, the mission is processed as successful and the alarm is dismissed; if the input is not correct, a re-entry may be requested.

Through this, the disclosure may induce an awakened state by requiring the user's attention to remember the provided sleep data and answer the question, and may assist in waking up more effectively than a simple alarm sound. Additionally, by generating the question based on the user's actual sleep data, a more personalized experience may be provided, thereby encouraging the user to participate more actively in the wake-up mission. Furthermore, by requiring the user to recall and respond to the data through the mission, it may contribute to cognitive and memory enhancement.

The snoring record menu is a menu that records and analyzes snoring using the microphone and AI sound detection function embedded in the user terminal. The main functions of the snoring record menu include recording the snoring occurrence time and duration in detail, visualizing the analysis results by time, and allowing the user to directly check the recorded snoring sound. Additionally, the snoring record menu may provide feedback to the user by linking the snoring data with the sleep score.

In some embodiments, when the snoring severity exceeds a reference value, the user terminal may record the snoring occurrence time, (snoring) duration, and snoring sound, and may calculate the sleep score based on the user's sleep duration, snoring occurrence time, and duration. The user terminal may provide the sleep duration, snoring occurrence time, duration, snoring sound, and sleep score through an alarm icon displayed on the screen.

The user terminal may calculate the sleep score by applying different weights for each sleep stage to the sleep duration, snoring occurrence time, and duration.

The user terminal divides the entire sleep duration into stages of light sleep (N1), intermediate sleep (N2), deep sleep (N3), and REM sleep, and calculates the sleep score that quantitatively evaluates the sleep quality by mapping the snoring occurrence information to each stage. In the disclosure, the user terminal divides the entire sleep into light sleep (N1), intermediate sleep (N2), deep sleep (N3), and REM sleep. However, the embodiment is not necessarily limited thereto, and the user terminal may calculate sleep stages using fewer or more stages.

The user terminal may apply differentiated weights by considering the impact of snoring on sleep quality in each sleep stage and calculate the sleep score accordingly. Specifically, the user terminal divides the user's total sleep duration into stages based on biometric signals or existing sleep rhythm data and tracks the snoring occurrence time and duration in real time in each sleep stage. Then, the user terminal may apply relatively lower weights to light sleep stages and higher weights to deep sleep and REM sleep stages and apply score deductions accordingly. For example, snoring that occurs during a deep sleep stage deteriorates the quality of sleep more severely than snoring of the same duration during a light sleep stage. Therefore, the user terminal may reflect this as a larger score deduction, thereby sensitively detecting degradation in sleep quality.

The user terminal may calculate the ratio of snoring duration to the total durations of each sleep stage and determine a total deduction coefficient by summing deduction coefficients obtained by multiplying the preset weight for each stage. The user terminal may calculate a total deduction score based on the total deduction coefficient and subtract it from a reference score (e.g., 100 points). In some embodiments, the result of frequency analysis may be reflected in the evaluation index, and snoring with high frequency or high intensity may act as a higher deduction factor.

In some embodiments, the user terminal may calculate a total deduction coefficient based on Equation 1.

$$D = {W}_{N 1} \cdot \frac{{S}_{N 1}}{{T}_{N 1}} + {W}_{N 2} \cdot \frac{{S}_{N 2}}{{T}_{N 2}} + {W}_{N 3} \cdot \frac{{S}_{N 3}}{{T}_{N 3}} + {W}_{REM} \cdot \frac{{S}_{REM}}{{T}_{REM}} \qquad [ Equation 1 ]$$

Here, D is a total deduction coefficient obtained by summing all the deduction coefficients, T_{N1}, T_{N2}, T_{N3}, and TREM represent the durations of each sleep stage (N1: light sleep, N2: intermediate sleep, N3: deep sleep, REM), S_{N1}, S_{N2}, S_{N3}, and S_{REM} represent the total durations of snoring that occurred during each sleep stage, and W_{N1}, W_{N2}, W_{N3}, and W_{REM} may represent deduction weights corresponding to each sleep stage. In some embodiments, W_{N1} may be smaller than W_{N2}, W_{N2} may be smaller than W_{N3}, and W_{N3} may be smaller than W_{REM}. However, the embodiment is not necessarily limited thereto.

In some embodiments, the user terminal may calculate the total deduction coefficient based on Table 1.

TABLE 1

| | | Snoring | | |
|---|---|---|---|---|
| Sleep | Time | duration | | |
| stage | (minutes) | (minutes) | Weight | Deduction Calculation |
| N1 | 60 | 5 | 0.5 | 0.5 * (5/60) = 0.0417 |
| N2 | 120 | 15 | 1.0 | 1.0 * (15/120) = 0.125 |
| N3 | 90 | 10 | 1.5 | 1.5 * (10/90) = 0.1667 |
| REM | 30 | 8 | 2.0 | 2.0 * (8/30) = 0.5333 |

That is, the user terminal may calculate the total deduction coefficient as 0.8667 (i.e., 0.0417+0.125+0.1667+0.5333), and may calculate a total deduction score based on the total deduction coefficient. The user terminal may calculate the total deduction score by multiplying the total deduction coefficient by a correction coefficient. The user terminal may calculate the sleep score based on the total deduction score.

The calculated sleep score may be provided through an alarm icon displayed on the user terminal's display, and when the icon is clicked or touched, the snoring occurrence time, duration of each occurrence, main sound data (e.g., acoustic spectrum or playable sample), snoring distribution by sleep stage, and the final calculated sleep score may be output in a summarized form. Accordingly, the user may take follow-up actions such as improving the sleep environment or receiving medical consultation based on the accumulated sleep analysis results.

Hereinafter, snoring detection and guidance on snoring reduction methods by severity through the snoring record menu will be described in detail.

FIG. 8 is a diagram illustrating a case in which a user terminal detects a snoring sound according to an embodiment.

Referring to FIG. 8, the user terminal 110 according to an embodiment may be an examinee device 110 to which the computing device 200 is applied, and may include a memory 210, at least one processor 220, a communication module 230, and a transceiver 240.

In addition, as described with reference to FIG. 2, the user terminal 110 may include a microphone as part of various sensors, and may include a user interface for input and output, such as a touch screen.

The user terminal 110 according to an embodiment may detect a snoring sound Z based on the output of a neural network model 1100 with respect to a sound S received through the microphone.

Specifically, the at least one processor 220 of the user terminal 110 may input the sound S received through the microphone into the neural network model 1100 stored in the memory 210, and may obtain the snoring sound Z as an output of the neural network model 1100.

At this time, the neural network model 1100 is a neural network trained in advance with a plurality of snoring sounds, and may output whether the input sound S corresponds to a snoring sound Z or whether the snoring sound Z is included in the input sound S.

The neural network model 1100 may be implemented using a convolutional neural network (CNN) or the like, and the type of model is not limited. In addition, the neural network model 1100 may include a plurality of layers, and weight values included in each layer may be updated according to the training.

As such, by using the neural network model 1100 that has been trained in advance with various types of snoring sounds, the user terminal 110 may detect the snoring sound Z of a user U with higher accuracy.

When the user terminal 110 detects the snoring sound Z of the user U, it may perform a frequency analysis on the snoring sound Z, determine a snoring severity, and guide a snoring reduction method according to the snoring severity.

Hereinafter, the frequency analysis of the snoring sound Z performed by the user terminal 110 will be described in detail.

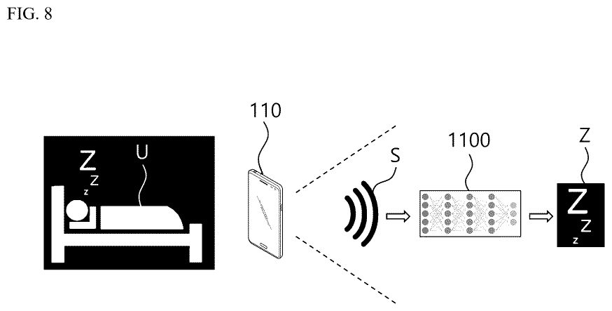

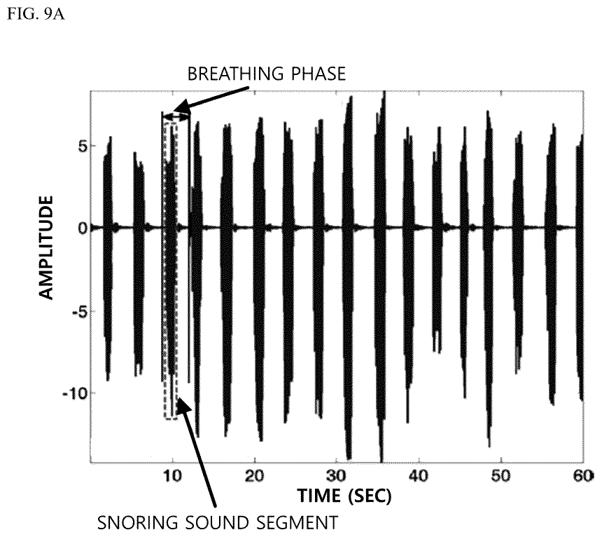

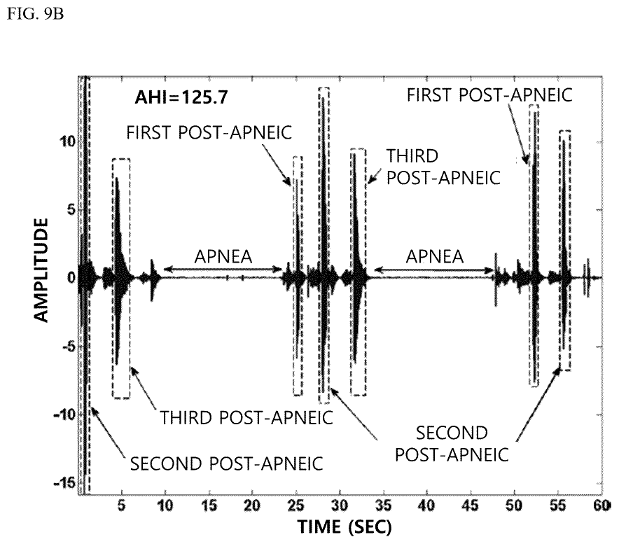

FIGS. 9 to 11 are diagrams illustrating cases in which a user terminal performs frequency analysis on snoring sound according to an embodiment.

Referring to FIGS. 9 to 11, at least one processor 220 of the user terminal 110 according to an embodiment may perform frequency analysis on the snoring sound Z.

Specifically, the at least one processor 220 may visualize time-frequency energy distribution and extract a specific frequency band using known signal processing techniques such as a spectrogram or fast Fourier transform (FFT) for the snoring sound Z.

Through this, the at least one processor 220 may obtain at least one among total variation norm, formant structure data, or peak intensity as a result of the frequency analysis on the snoring sound Z.

For example, the at least one processor 220 may determine whether the total variation of the snoring sound Z is high or low based on the frequency analysis result.

At this time, the total variation of the snoring sound Z may vary depending on the severity of obstructive sleep apnea (OSA). As shown in FIG. 9A, the snoring sound Z of a user who does not have obstructive sleep apnea is relatively consistent over time and has low total variation in the frequency domain, whereas, as shown in FIG. 9B, the snoring sound Z of a user with obstructive sleep apnea shows high variability over time, resulting in high total variation in the frequency domain.

As shown in FIG. 9B, the snoring sound Z of a user with obstructive sleep apnea may occur irregularly in three bursts following a period of apnea. Accordingly, the total variation of the snoring sound Z in the frequency domain becomes inevitably high.

Therefore, the at least one processor 220 may determine that the snoring severity increases as the total variation of the snoring sound Z increases.

Additionally, the at least one processor 220 may determine at least one of formant structure data or peak intensity based on the frequency analysis result of the snoring sound Z.

At this time, a formant refers to a resonance frequency generated by the anatomical structures of the upper airway such as the pharynx, oral cavity, and nasal cavity. Snoring sounds also have resonance characteristics in specific frequency bands depending on upper airway structures such as the soft palate and tongue root.

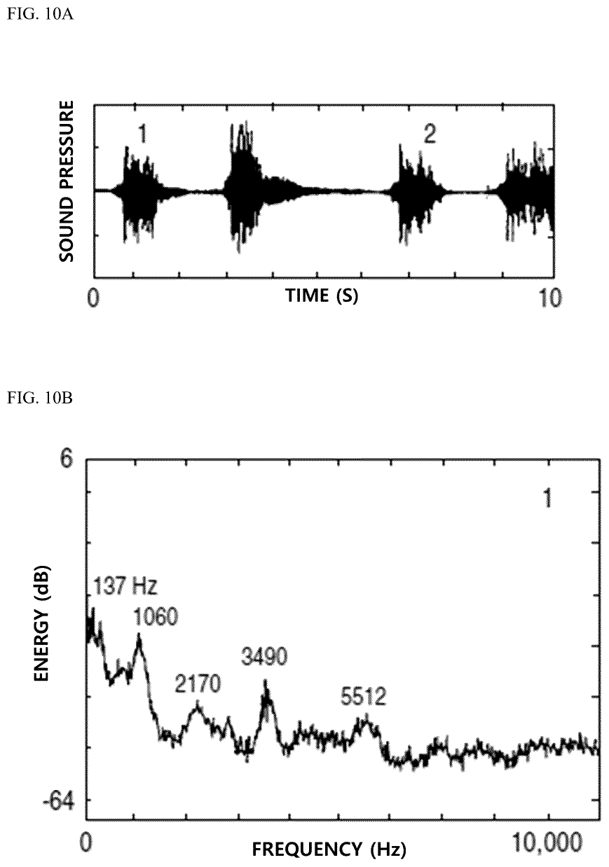

Accordingly, the at least one processor 220 may convert the snoring sound Z in the time domain as shown in FIG. 10A into energy distribution in the frequency domain as shown in FIG. 10B, identify resonance frequencies (numerically indicated in the figure) of the snoring sound Z, and generate formant structure data.

As such, the at least one processor 220 may analyze the pattern of upper airway obstruction using the formant structure data, which is quantized information on the frequency-based resonance characteristics (formants) of the snoring sound Z, and determine the snoring severity.

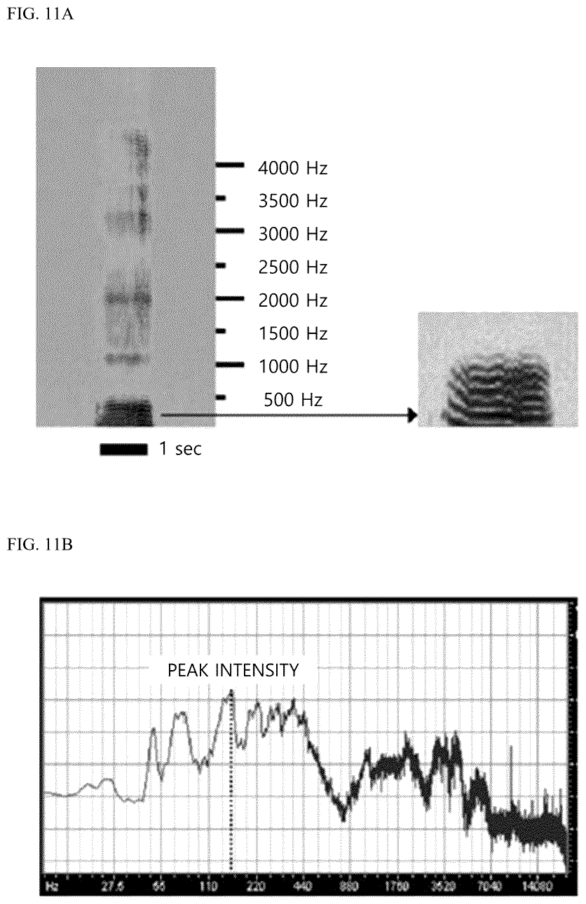

In addition, the snoring sound Z of a primary snorer may show peak intensity between 100 Hz and 300 Hz, as illustrated in FIGS. 11A and 11B, whereas the snoring sound Z of a user with sleep apnea may exhibit peak intensity above 1,000 Hz.

Thus, the at least one processor 220 may determine the snoring severity based on the peak intensity in the frequency domain of the snoring sound Z.

FIGS. 9 to 11 describe how the at least one processor 220 of the user terminal 110 performs frequency analysis based on signal processing of the snoring sound Z.

However, in some embodiments, the user terminal 110 may also determine the snoring severity corresponding to the snoring sound Z using a neural network model trained on the correlation between a plurality of snoring sounds and snoring severities.

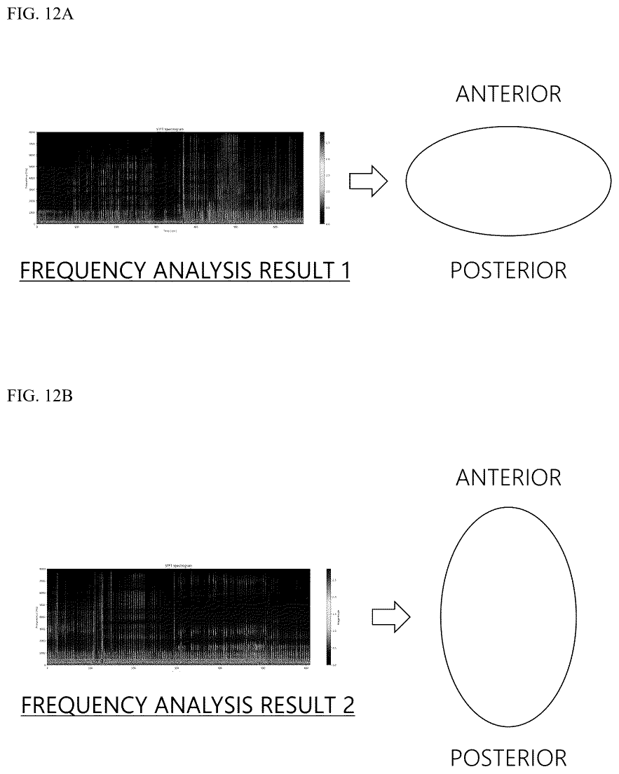

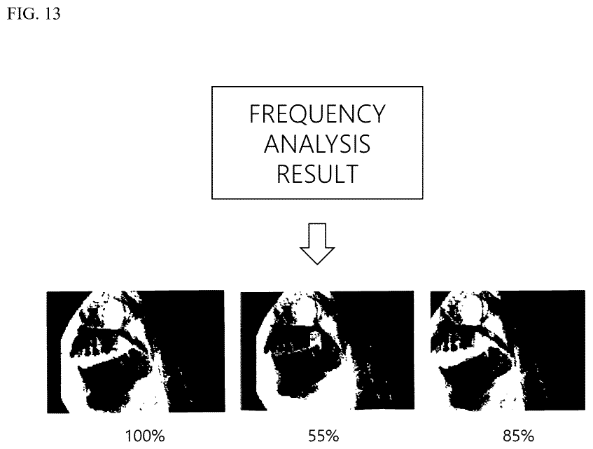

FIGS. 12A and 12B are diagrams illustrating cases in which a user terminal estimates a shape of an airway according to an embodiment, and FIG. 13 is a diagram illustrating a case in which a user terminal estimates an obstruction level of an airway according to an embodiment.

Referring to FIG. 12, at least one processor 220 according to an embodiment may determine a snoring severity based on the result of frequency analysis on the snoring sound Z.

For example, the snoring severity may be classified using an index such as the Apnea-Hypopnea Index (AHI), which indicates the sum of the number of apneas and hypopneas per hour of sleep.

For example, the at least one processor 220 may estimate the shape of the airway of the user U based on the result of the frequency analysis, and may determine the snoring severity based on the estimated airway shape.

In general, when the airway has an elliptical shape with a long axis along the anteroposterior axis (long axis in the sagittal plane), as shown in FIG. 12B, the possibility of airway obstruction is higher compared to when the airway has an elliptical shape with a long axis in the lateral direction (long axis in the coronal plane), as shown in FIG. 12A. In such cases, the likelihood of developing obstructive sleep apnea is increased.

Accordingly, the at least one processor 220 may estimate that the airway has an elliptical shape with a long axis in the anteroposterior direction when the total variation of the snoring sound Z is higher, when the formant structure data of the snoring sound Z indicates upper airway obstruction, and when the peak intensity of the snoring sound Z is located in a higher frequency band.

The at least one processor 220 may determine that the snoring severity of the user U is higher when the airway has an elliptical shape with a long axis in the anteroposterior direction.

In addition, referring to FIG. 13, the at least one processor 220 may estimate the obstruction level of the airway of the user U based on the result of the frequency analysis, and may determine a snoring severity based on the obstruction level of the airway. For example, the frequency analysis result may include spectrogram data, but embodiments are not necessarily limited thereto.

Specifically, the at least one processor 220 may estimate that the obstruction level of the airway is relatively high as the total variation of the snoring sound Z increases, as the formant structure data of the snoring sound Z indicates upper airway obstruction, and as the peak intensity of the snoring sound Z is located in a higher frequency band.

The at least one processor 220 may determine that the snoring severity of the user U is higher as the obstruction level of the airway increases.

The at least one processor 220 according to an embodiment may independently estimate both the airway shape and the obstruction level by performing signal processing and analysis with the snoring sound Z as an input. More specifically, the at least one processor 220 is included in the user terminal, and the at least one processor 220 converts the snoring sound Z received through the microphone into a frequency domain and evaluates the airway shape (e.g., structural characteristic) and functional obstruction level based on the result of frequency analysis.

The at least one processor 220 may estimate the airway shape based on time-frequency energy distribution of the snoring sound Z, i.e., spectrogram analysis. The spectrogram visualizes energy distribution along the time and frequency axes. The at least one processor 220 analyzes the occurrence position and distribution of formants reflecting the resonance characteristics of the airway from the distribution. At this time, the at least one processor 220 may define the duration of energy in the time axis as the anteroposterior axis length, and the lateral dispersion of frequency or energy as the left-right axis length.

The at least one processor 220 may calculate an airway shape index, i.e., an elliptical ratio of the airway, by computing the ratio of the anteroposterior axis length to the left-right axis length. If the airway shape index is greater than or equal to a reference value, the at least one processor 220 may determine that the airway is an anatomically high-risk structure for obstruction with an elliptical shape elongated in the anteroposterior direction in the sagittal plane. Conversely, if the elliptical ratio is relatively low, the at least one processor 220 may determine that the airway is laterally widened in the coronal plane and may evaluate it as a relatively open airway.

In addition, the at least one processor 220 may determine at least one of total variation, formant structure data, or peak intensity based on the result of the frequency analysis. The total variation is defined as the rate of change in frequency energy over time and increases as the sound fluctuates more irregularly. The at least one processor 220 reflects the phenomenon of repeated airway closure and opening, and may determine that higher total variation indicates a higher degree of obstruction.

The formant concentration level is a value that quantifies whether energy is concentrated in a specific resonance frequency band. A high concentration level may be associated with resonance characteristics generated from a narrowed upper airway structure. The at least one processor 220 may estimate that the obstruction level is severe when the formant concentration level is relatively high.

The peak intensity band indicates the location of the highest intensity in the energy spectrum. The at least one processor 220 may determine that when the peak intensity is located in a high-frequency range, the snoring has more pathological characteristics and more severe airway obstruction.

The at least one processor 220 may normalize the three indicators and compute a weighted sum to derive a single obstruction score. The at least one processor 220 may classify the obstruction level by comparing the score with a reference value. For example, the at least one processor 220 may classify the severity into categories such as mild or severe based on the comparison result, and the classification result may be used as a criterion for selecting a snoring reduction solution or recommending treatment to the user.

As such, by analyzing the time-frequency information included in the snoring sound Z from multiple perspectives, the at least one processor 220 may independently evaluate the airway shape through the anteroposterior/left-right axis ratio, and the obstruction level of the airway through the total variation, formant concentration level, and peak intensity band, thereby implementing a personalized snoring diagnosis and management system for the user.

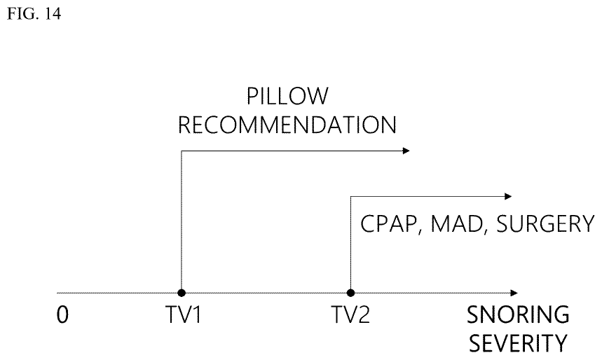

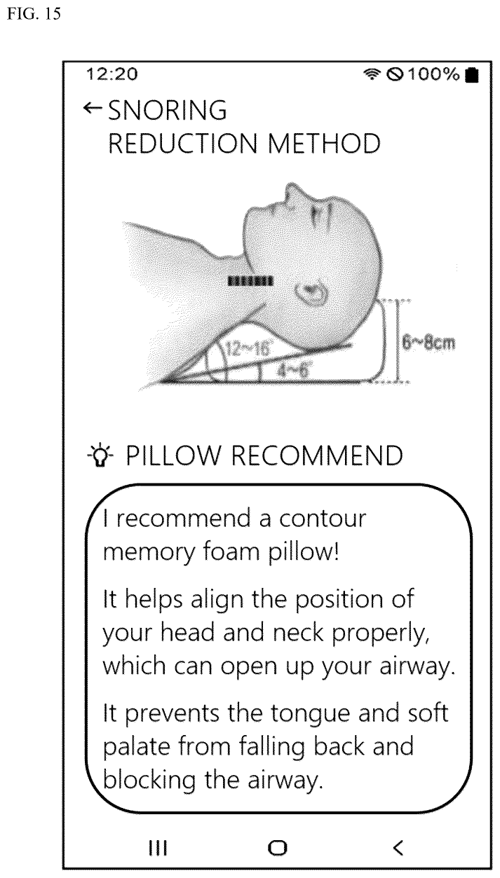

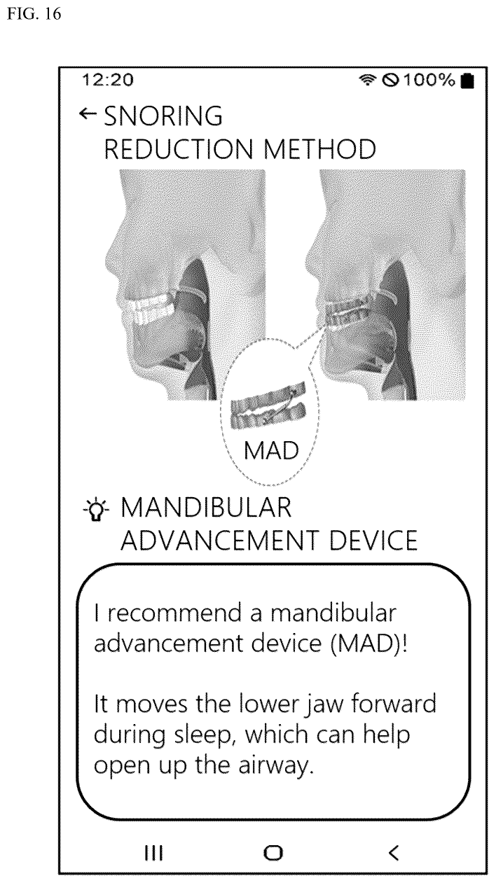

FIG. 14 is a diagram illustrating a case in which a user terminal outputs a snoring reduction method according to an embodiment, and FIGS. 15 to 16 are diagrams illustrating cases in which a user terminal outputs a message guiding a snoring reduction method according to an embodiment.

Referring to FIG. 14, the at least one processor 220 according to an embodiment may output a message guiding a snoring reduction method when the snoring severity is equal to or greater than a preset value.

At this time, the at least one processor 220 may control the user interface of the user terminal 110 to output the message, and through this, the user U may be guided with the snoring reduction method.

For example, as illustrated in FIGS. 14 and 15, when the snoring severity is equal to or greater than a first threshold value TV1, the at least one processor 220 may output a message recommending a pillow for head and neck alignment.

At this time, the at least one processor 220 may recommend at least one of a latex pillow, a memory foam pillow, a wedge type pillow, a contour type pillow, or a body pillow as the pillow for head and neck alignment.

When the alignment of the head and neck is supported through the pillow, the obstruction level of the airway may be alleviated and snoring reduction may be achieved. This may be informed to the user U to enhance the user's convenience.

In addition, as illustrated in FIGS. 14 and 16, when the snoring severity is equal to or greater than a second threshold value TV2, the at least one processor 220 may output a message recommending a continuous positive airway pressure (CPAP) device, a mandibular advancement device (MAD), or surgical treatment. In some embodiments, the at least one processor 220 may also recommend at least one pillow for head and neck alignment.

At this time, the second threshold value TV2 is greater than the first threshold value TV1, and the user terminal 110, in cases where the snoring severity is high, may go beyond simply recommending a pillow and present medical devices or surgical treatment options to help alleviate the user's snoring.

As such, the user terminal 110 may present an optimized solution for the user by guiding stepwise reduction methods according to the snoring severity.

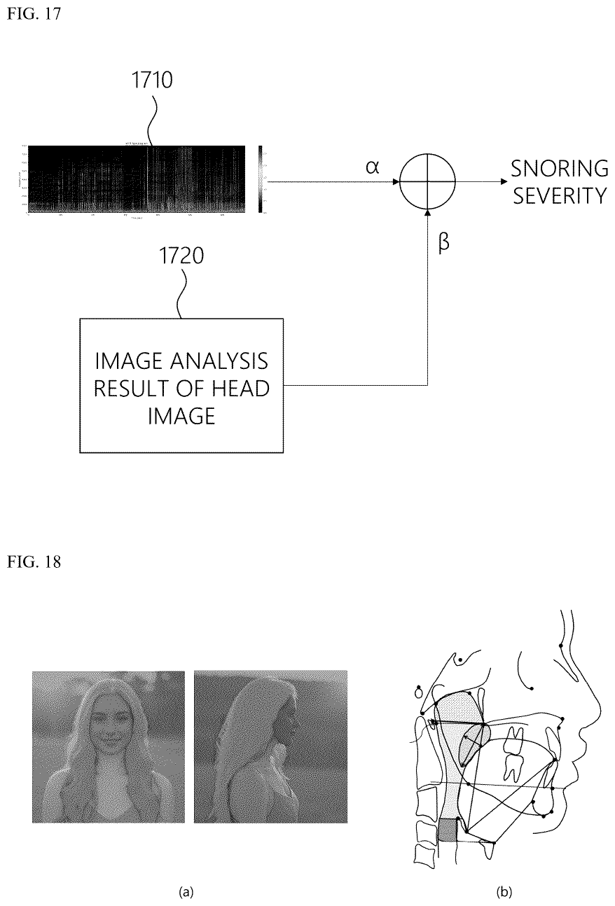

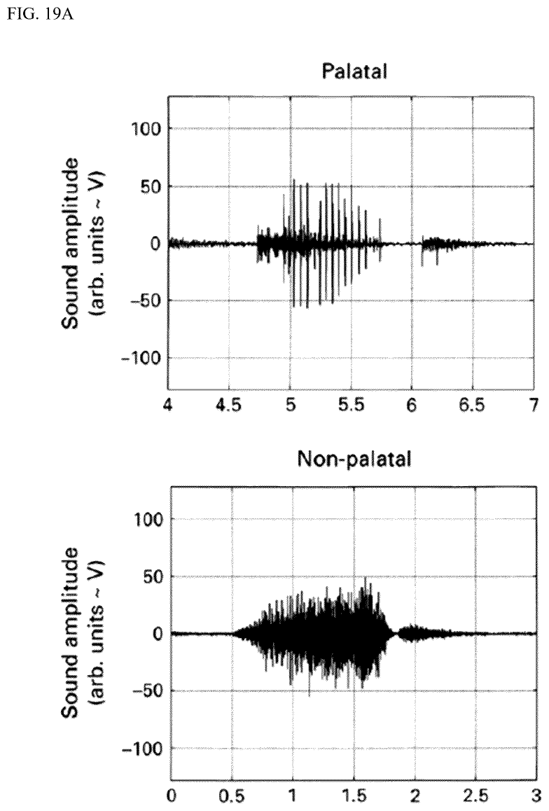

FIG. 17 is a diagram illustrating a case in which a user terminal determines a snoring severity by additionally using a head image according to an embodiment, and FIG. 18 is a diagram illustrating an example of a head image.

Referring to FIG. 17, the at least one processor 220 according to an embodiment may determine a snoring severity based on a frequency analysis result 1710 of the snoring sound Z and an image analysis result 1720 of a head image.

For example, the at least one processor 220 may determine the snoring severity based on the result of summing: a value obtained by applying a weight α to the snoring severity calculation result based on the frequency analysis result 1710 of the snoring sound Z; and a value obtained by applying a weight β to the snoring severity calculation result based on the image analysis result 1720 of the head image. At this time, the at least one processor 220 may use a snoring severity table corresponding to the sum result, and the weights a and β may be preset or adjustable by the user.

The head image may include at least one of a facial image (FIG. 18(a)), a lateral cephalometric radiograph (FIG. 18(b)), or a lateral magnetic resonance image.

The user terminal 110 may receive a head image of the user U from an external source, and in some embodiments, may obtain the head image through a camera embedded in the user terminal 110.

In general, patients with obstructive sleep apnea have facial characteristics such as increased facial height, a retruded mandible, and a reduced projection and width of the nose.

Accordingly, the at least one processor 220 may determine that the snoring severity is higher when the face appears longer, the nose is smaller, and the chin is retracted in the result of image processing of the facial image shown in FIG. 18(a).

In addition, in patients with obstructive sleep apnea, the pharyngeal airway space is narrowed, the shape of the pharynx is circular or elliptical with the long axis located in the sagittal plane, the airway shape is also elliptical with the long axis in the sagittal plane, the anterior facial height is increased, and the hyoid bone may be positioned lower.

Accordingly, the at least one processor 220 may determine that the snoring severity is higher when, based on the image processing result of a lateral cephalometric radiograph (including a computed tomography image) shown in FIG. 18(b) or a lateral magnetic resonance image, the pharyngeal airway space is narrowed, the shape of the pharynx is circular or elliptical with the long axis in the sagittal plane, the airway shape is elliptical with the long axis in the sagittal plane, the anterior facial height is increased, and the hyoid bone is located lower.

As such, in some embodiments, the user terminal 110 may improve the prediction accuracy of disease and condition by performing frequency analysis on the snoring sound Z in parallel with image processing on the head image.

FIGS. 17 and 18 describe how the at least one processor 220 of the user terminal 110 derives a snoring severity based on image processing of the head image.

However, in some embodiments, the user terminal 110 may determine the snoring severity corresponding to a head image using a neural network model trained on the correlation between the head image and snoring severity. The user terminal 110 according to an embodiment is configured to more precisely determine a snoring severity by integrally utilizing the frequency analysis result of the snoring sound and the image analysis result of the user's head image. In this case, the frequency analysis result may include various analysis indicators such as total variation, formant concentration level, peak intensity band, and the like. The user head image may include a facial image and a lateral medical image of the head, wherein the lateral medical image may include a lateral cephalometric radiograph (including the computed tomography image) or a lateral magnetic resonance image.

The analysis result of the facial image may include external features such as facial height, nasal projection, and chin position. In addition, the analysis result of the lateral medical image may include quantitative anatomical indicators such as airway shape, airway cross-sectional area, position of the hyoid bone, anterior facial height, and the like. The at least one processor of the user terminal 110 may apply a preset weight to each of the analysis indicators to finally calculate the snoring severity.

For example, the user terminal 110 may apply a first weight to the frequency analysis result, a second weight to the facial image analysis result, and a third weight to the lateral medical image analysis result, considering the reliability and contribution of each information source to the determination of severity. In this case, the third weight may be set greater than the second weight, and the second weight may be set greater than the first weight, so that the more reliable anatomical information may be more heavily reflected in the determination of severity. This is based on the medical rationale that the pathological cause of sleep apnea is mainly due to pharyngeal airway narrowing or anatomical deformation, and that the information obtained through lateral medical images most directly reflects the cause. However, the embodiment is not necessarily limited thereto.

The facial image analysis, as an indicator indirectly reflecting anatomical structure, has a medium level of reliability, and the frequency analysis result, as an indirect sound-based indicator, is assigned a relatively lower weight. This weight-based integrated determination method allows for correction of uncertain input values affected by external conditions and may provide accurate and consistent severity output. In addition, the processor may apply the weight to each analysis score from each information source, perform weighted summation, and provide the severity result to the user by referring to a preconfigured severity mapping table based on the summation result.

Furthermore, the weight values may be dynamically adjusted according to user settings or an AI learning algorithm, thereby enabling user-personalized severity determination. As such, the disclosure provides a technical effect of enabling accurate diagnosis of an individual user's snoring condition by integrating various input data with different levels of reliability and analytical characteristics, and by applying a weight-based fusion approach. As a result, the disclosure enables the implementation of an advanced user terminal 110 that may enhance both the accuracy and personalization of sleep health management.

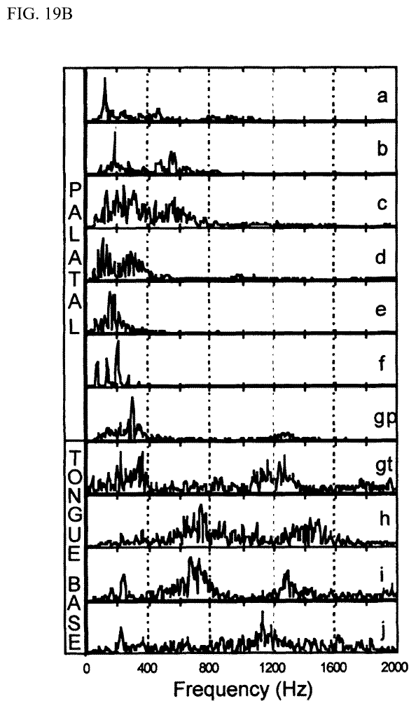

FIGS. 19A and 19B are diagrams illustrating cases in which a user terminal determines a snoring source location based on frequency analysis of snoring sound according to an embodiment.

Referring to FIGS. 19A and 19B, the at least one processor 220 may determine the snoring source location based on the result of the frequency analysis of the snoring sound Z and may output a message guiding the snoring source location.

In general, as illustrated in FIG. 19A, snoring originating from the soft palate (palatal) has a waveform in the form of impulses and is characterized by low-frequency sound with a relatively narrow frequency range, as illustrated in FIG. 19B.

Also, as shown in FIG. 19A, snoring originating from locations other than the soft palate (for example, the tongue base) has a waveform without impulses, and as shown in FIG. 19B, is characterized by high-frequency sound with a broader frequency range.

Accordingly, the at least one processor 220 may determine and guide that the snoring source location is the soft palate when the pitch of the snoring sound Z is within a low-frequency range below 500 Hz. On the other hand, if the pitch of the snoring sound Z exhibits sporadic energy distribution in a higher frequency band, the at least one processor 220 may determine and guide that the snoring source location is a region other than the soft palate (e.g., the tongue base).

As such, in some embodiments, the user terminal 110 may improve the accuracy of disease and condition prediction by predicting the snoring source location based on frequency analysis of the snoring sound Z.

Hereinafter, a control method of the user terminal 110 according to an embodiment will be described. The control method of the user terminal 110 described below may be applied to the user terminal 110 according to the above-described embodiments. Therefore, unless otherwise specified, the contents described above with reference to FIGS. 8 to 19 may equally apply to the control method of the user terminal 110 according to an embodiment.

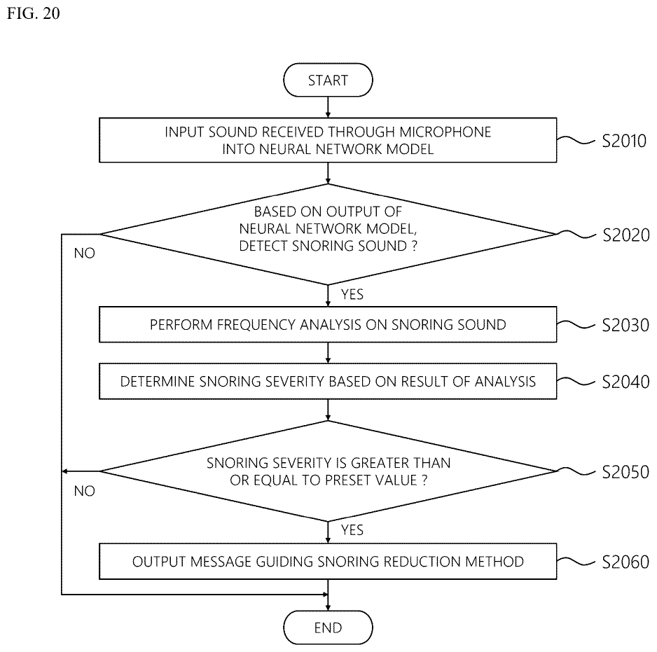

FIG. 20 is a flowchart illustrating a case of guiding a snoring reduction method as part of a control method for a user terminal according to an embodiment.

Referring to FIG. 20, the user terminal 110 according to an embodiment may input a sound S received through a microphone into a neural network model 1110 (S2010).

Specifically, the at least one processor 220 of the user terminal 110 may input the sound S received through the microphone into the neural network model 1100 stored in the memory 210 and may obtain a snoring sound Z as the output of the neural network model 1110.

When the user terminal 110 according to an embodiment detects the snoring sound Z based on the output of the neural network model 1110 (YES in step 2020), the user terminal 110 may perform frequency analysis on the snoring sound Z (S2030) and may determine a snoring severity based on the result of the analysis (S2040).

Specifically, the user terminal 110 may visualize energy distribution across time and frequency using known types of signal processing techniques such as a spectrogram and fast Fourier transform (FFT), and may extract specific frequency bands.

Through this, the user terminal 110 may obtain at least one of total variation norm, formant structure data, or peak intensity as a result of the frequency analysis of the snoring sound Z.

The user terminal 110 may determine that a snoring severity increases as the total variation of the snoring sound Z increases. The user terminal (110) may determine a snoring severity by analyzing the pattern of upper airway obstruction using formant structure data, which represents quantified information of the resonance characteristics (formants) of the snoring sound (Z) in the frequency domain. The user terminal (110) may determine a snoring severity based on the peak intensity of the snoring sound (Z) in the frequency domain.

When the snoring severity is greater than or equal to a preset value (YES in step 2050), the user terminal 110 may output a message guiding a snoring reduction method (S2060).

For example, when the snoring severity is greater than or equal to a first threshold value, the user terminal 110 may output a message recommending a pillow for head and neck alignment.

At this time, the user terminal 110 may recommend at least one of a latex pillow, a memory foam pillow, a wedge type pillow, a contour type pillow, or a body pillow as a pillow for head and neck alignment.

When alignment of the head and neck is supported by the pillow, the airway obstruction level may be alleviated, and snoring reduction may be achieved. This may be guided to the user U to enhance convenience.

In addition, when the snoring severity is greater than or equal to a second threshold value, the user terminal 110 may output a message recommending a continuous positive airway pressure (CPAP) device, a mandibular advancement device (MAD), or surgical treatment.

Here, the second threshold value is greater than the first threshold value. In cases of severe snoring severity, the user terminal 110 may provide options beyond pillow recommendation, such as medical devices or surgical procedures, to help alleviate snoring in the user U.

As such, the user terminal 110 may present an optimized solution for the user by guiding stepwise reduction methods according to the snoring severity.

The above describes in detail detecting snoring and guiding snoring reduction methods based on severity.

Next, regarding the menu structure, the sleep therapy menu may provide more than 48 types of sound therapy patterns to induce sleep for the user. When “Pure Binaural Beats” is selected, the user may be guided to a screen for selecting sleep-inducing audio (mp3) data. The available audio includes, for example, Delta Wave and Relaxation Beats, and may be provided according to the user's sleep state and preferences. In addition, the sound therapy execution screen may include a user-friendly UI where the user may select a playback button, playback duration (3 minutes/30 minutes), repeat mode, etc.

The focus category provides various functions for improving the user's work concentration, rest state, and mental stability through a mindfulness feature. Key submenus may include a work therapy menu, a rest therapy menu, and a mindfulness menu.

The work therapy menu is designed to help the user maintain an optimal concentration state during tasks. Its main functions may include providing sound therapy to enhance concentration according to the user's state, setting work durations, and providing feedback on concentration level to support improved work efficiency.

The rest therapy menu may provide relaxation sound therapy that helps reduce fatigue and relieve tension in the user. In addition to sound therapy, the rest mode menu may combine specific patterns of EEG stimulation (tES) to maximize psychological stabilization effects.

The mindfulness menu may provide meditation and breathing therapy to stabilize the user's mind. It may provide mindfulness content along with audio data and offer customized guides for meditation or breathing practice.

The recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise.

Although the above-described embodiments have been described as utilizing aspects of the presently invented subject matter in one or more stand-alone computer systems, the disclosure is not limited thereto and may also be implemented in conjunction with any computing environment such as networks or distributed computing environments. Furthermore, aspects of the subject matter of the disclosure may be implemented across multiple processing chips or devices, and storage may be similarly affected across multiple devices. Such devices may include PCs, network servers, and portable devices.

While the disclosure has been described in connection with certain embodiments, various modifications and alterations may be made without departing from the scope of the invention as understood by those skilled in the art to which the invention pertains. Such modifications and alterations should be regarded as falling within the scope of the claims appended hereto.

The effects provided in the disclosure are not limited to those described above but may be extended to various aspects that can be derived from some embodiments described below.

Meanwhile, although the disclosure has been described in connection with certain embodiments, various modifications and changes may be made without departing from the scope of the invention as understood by those skilled in the art to which the invention pertains. Such modifications and changes should be regarded as falling within the scope of the claims appended to the disclosure.

What is claimed is:

1. A personalized biofeedback providing method comprising:
obtaining, through at least one of a terminal and a wearable biosensor, first information corresponding to at least one of Heart Rate Variability (HRV) information, electroencephalogram (EEG) information, heart rate information, temperature information, stress information, body composition information, weight information, oxygen saturation information, pulse information, blood pressure information, iris information, voice information, vein information, and electrocardiogram (ECG) information of a user;
deriving a difference by comparing a first circadian rhythm, which is calculated based on a time point at which the first information is obtained through the terminal, with a preset reference circadian rhythm; and
delivering a stimulus to a brain of the user to entrain synchronized oscillations in a plurality of regions of the brain of the user so that the first circadian rhythm matches the reference circadian rhythm, based on at least one of the terminal and a stimulation device,
wherein the stimulus is one of:
a first stimulus for entraining synchronized gamma oscillations in the plurality of regions of the brain of the user,
a second stimulus for entraining synchronized delta, theta, alpha, or beta oscillations in the plurality of regions of the brain of the user, or
a third stimulus that combines the first stimulus and the second stimulus.

2. The personalized biofeedback providing method of claim 1,
wherein the reference circadian rhythm is divided into a wake-up zone within a first hour after waking up of the user, a sleep zone within a second hour before going to sleep of the user, and a daily zone excluding the wake-up zone and the sleep zone based on 24 hours, and
wherein the difference varies depending on which of the divided zones the time point at which the first information is obtained falls into.

3. The personalized biofeedback providing method of claim 2,
wherein, when the EEG of the user is entrained by the stimulus, at least one of a parasympathetic nerve and a sympathetic nerve of the user is accelerated or decelerated by EEG synchronization, whereby at least one of the HRV, temperature, and blood pressure of the user is changed so that the first circadian rhythm matches the reference circadian rhythm.

4. The personalized biofeedback providing method of claim 3,
wherein, when the first stimulus and the second stimulus are delivered together to the brain of the user, a region in which entrainment occurs is expanded, so that the time required to make the first circadian rhythm match the reference circadian rhythm is shortened, and
wherein when transcranial Alternating Current Stimulation (tACS) is applied as one of electrical stimulation of the first stimulus,
the tACS is a first combined signal in which ON/OFF is repeated according to a preset first frequency, and a signal that is ON according to the first frequency is modulated as a stimulus according to a preset second frequency.

5. The personalized biofeedback providing method of claim 3,
wherein, when the first stimulus and the second stimulus are delivered together to the brain of the user,
a region in which entrainment occurs is expanded, so that the time required to make the first circadian rhythm match the reference circadian rhythm is shortened, and
wherein when a binaural beats stimulus is applied as one of sound stimulations of the second stimulus, the binaural beats stimulus induces the entrainment through an auditory phenomenon in which the brain of the user recognizes a new frequency corresponding to a frequency difference by hearing sounds of different frequencies in both ears of the user.

6. The personalized biofeedback providing method of claim 2,
wherein, when the time point at which the first information is obtained is the wake-up zone, an alpha wave of 8 to 13 Hz is applied as the stimulus,
wherein when the time point at which the first information is obtained is the sleep zone, a delta wave of less than 4 Hz is applied as the stimulus,
and wherein when the time point at which the first information is obtained is the daily zone, at least one of the alpha wave of 8 to 13 Hz, the beta wave of 14 to 29 Hz, and the gamma wave of 30 to 100 Hz is applied as the stimulus.

7. The personalized biofeedback providing method of claim 1, further comprising:
performing a wake-up mission of sound detection through a user terminal; and
terminating an alarm of the user terminal when a sound corresponding to a preset sound is generated as a result of AI-based recognition of at least one sound among a clap sound, a laughing sound, and a speech sound of the user through the user terminal during the wake-up mission of sound detection.

8. The personalized biofeedback providing method of claim 1, further comprising:
performing a wake-up mission of hand gesture recognition through a user terminal; and
terminating an alarm of the user terminal when a preset specific action is taken based on recognition of a hand gesture of the user captured through the user terminal using Mediapipe-based gesture AI during the wake-up mission of hand gesture recognition.

9. The personalized biofeedback providing method of claim 1, further comprising:
performing a wake-up mission of solving a math problem through a user terminal; and
terminating an alarm of the user terminal when a preset correct answer is input based on recognition of input data entered through the user terminal using MLKit-based digital ink recognition AI during the wake-up mission of solving the math problem.

10. The personalized biofeedback providing method of claim 1, further comprising:
performing a wake-up mission of answering a sleeping information quiz through a user terminal; and
terminating an alarm of the user terminal when the input data entered through the user terminal corresponds to a preset correct answer for a question generated based on sleep record data of the user during the wake-up mission of answering the sleeping information quiz.

11. The personalized biofeedback providing method of claim 1, further comprising:
analyzing circadian rhythm data of a user, and providing customized management feedback classified into a sleep mode, a wake-up mode, and a daily management mode based on a result of the analysis; or
analyzing the circadian rhythm data of the user, calculating an optimal sleep start time and an optimal wake-up time for a next day based on an average sleep duration, a sleep start time, and a wake-up time pattern of the user, and providing a notification or recommendation message of the calculation result to a user terminal.

12. The personalized biofeedback providing method of claim 1, further comprising:
presenting at least one sound therapy track for each sound therapy mode and executing a sound therapy track selected by the user; and
presenting at least one piece of preset traditional fairy tale data and analyzing a traditional fairy tale selected by the user based on AI to provide the tale in a TTS (text-to-speech) service format.

13. The personalized biofeedback providing method of claim 1, further comprising:
measuring a real-time concentration state of a user, classifying the concentration state into three stages, and automatically selecting and adjusting customized therapy content composed of at least one combination of sound therapy and electrical stimulation (tES) according to each of the stages to provide to the user.

14. The personalized biofeedback providing method of claim 1, further comprising:
detecting snoring data of a user, and visualizing a snoring occurrence time to provide a notification to a user terminal.

15. The personalized biofeedback providing method of claim 14,
wherein the detecting the snoring data of the user and visualizing the snoring occurrence time to provide the notification to the user terminal comprises:
providing snoring data to the user terminal through a notification icon displayed on the user terminal when snoring is detected.

16. The personalized biofeedback providing method of claim 1, further comprising:
detecting a snoring sound based on an output of a neural network model for a sound received through a microphone;
determining a snoring severity based on a frequency analysis result of the snoring sound; and
providing a snoring reduction method when the snoring severity exceeds a preset value.

17. The personalized biofeedback providing method of claim 16, further comprising:
determining an anteroposterior length and lateral length based on the frequency analysis result, and estimating an airway shape based on the anteroposterior length and the lateral length;
determining a total variation norm, a formant concentration level, and a peak intensity position based on the frequency analysis result;
estimating an obstruction level of the airway based on the total variation norm, the formant concentration level, and the peak intensity position; and
determining the snoring severity based on the airway shape and the obstruction level.

18. The personalized biofeedback providing method of claim 16,
wherein the determining the snoring severity based on the frequency analysis result of the snoring sound comprises:
determining the snoring severity based on the frequency analysis result of the snoring sound and an image analysis result for a head image of the user, and
wherein the head image comprises a facial image and a cranial medical image.

19. The personalized biofeedback providing method of claim 18,
wherein the determining the snoring severity based on the frequency analysis result of the snoring sound and the image analysis result for the head image comprises:
applying a first weight to the frequency analysis result, applying a second weight to the analysis result of the facial image, and applying a third weight to the cranial medical image to determine the snoring severity, and
wherein the third weight is greater than the second weight, and
the second weight is greater than the first weight.

20. The personalized biofeedback providing method of claim 16, further comprising:
recording a snoring occurrence time, duration, and snoring sound when the snoring severity exceeds a reference value;
calculating a sleep score by applying different weights for each sleep stage to a user's sleep duration, the snoring occurrence time, and the duration; and
providing the user's sleep duration, the snoring occurrence time, the duration, the snoring sound, and the sleep score through an alarm icon displayed on a screen.
